(19) United States
(12) Patent Application Publication
(10) Pub. No.: US 2019/0256499 A1
(43) Pub. Date: Aug. 22, 2019

(54) SUBSTITUTED QUINOXALINE DERIVATIVES AS INHIBITORS OF PFKFB

(71) Applicant: Merck Patent GmbH, Darmstadt (DE)

(72) Inventor: Hans-Peter Buchstaller, Griesheim (DE)

(73) Assignee: Merck Patent GmbH, Darmstadt (DE)

(21) Appl. No.: 16/347,830

(22) PCT Filed: Nov. 6, 2017
(86) PCT No.: PCT/EP2017/078263

§ 371 Date: May 7, 2019

(30) Foreign Application Priority Data
Nov. 8, 2016 (EP) 16197783.0

(51) Int. Cl. C07D 403/14; A61P 35/00; A61K 9/00; A61K 9/02; A61K 9/20; A61K 9/28; A61K 9/48; C07D 471/04; C07D 405/14; C07D 401/14

(52) U.S. Cl. CPC C07D 403/14; A61P 35/00; A61K 9/0019; A61K 9/02; A61K 9/0048; C07D 401/14; A61K 9/2826; A61K 9/4825; C07D 471/04; C07D 405/14; A61K 9/2095

(57) ABSTRACT

Substituted quinoxaline derivatives are useful for the prevention and/or treatment of medical conditions known as hyperproliferative diseases. The compounds are also useful for medical conditions that are affected by inhibiting 6-phosphofructo-2-kinase/fructose-2,6-bisphosphatase (PFKFB). Further, the compounds are useful for the prevention and/or treatment of cancer.

The present invention relates to substituted quinoxaline derivatives. These compounds are useful for inhibiting 6-phosphofructo-2-kinase/fructose-2,6-bisphosphatase (PFKFB) and for the prevention and/or treatment of medical conditions affected by PFKFB activity. They are in particular useful for the prevention and/or treatment of cancer diseases.

### BACKGROUND OF THE INVENTION

Glycolysis is a non-oxidative metabolic pathway in which glucose is degraded by cells to generate ATP (adenosine triphosphate), i.e. energy. While normal, i.e. healthy cells are usually favoring this pathway for generating ATP only under anaerobic conditions, many cancer cells generate ATP—even in the presence of oxygen—from glucose via glycolysis; the glycolytic rate can be up to 200 times greater in malignant rapidly-growing tumor cells than in healthy cells. This switch of energy metabolism in cancer cells to the process of “aerobic glycolysis” is known as the “Warburg Effect” (D. G. Brooke et al., Biorganic & Medicinal Chemistry 22 (2014) 1029-1039; T. V. Pyrkov et al., Chem Med Chem 2013, 8, 1322-1329).

The rate of glycolysis is regulated by several enzymes, including phosphofructokinase, that catalyze irreversible reactions in the course of glycolysis. 6-phosphofructo-1-kinase (PFK-1), the precursor of anaerobic ATP production, which converts fructose-6-phosphate (F6P) to fructose-1,6-bisphosphate (F1,6-BP), is considered to be the rate-limiting enzyme in the process of converting glucose into pyruvate. PFK-1 is allosterically activated by fructose-2,6-bisphosphate (F2,6-BP) which is synthesized from F6P by phosphofructokinase-2 (PFK-2; 6-phosphofructo-2-kinase/fructose-2,6-bisphosphatase, PFKFB). Four isoforms of the PFK-2 family are known, namely PFKFB1, PFKFB2, PFKFB3, and PFKFB4 (D. G. Brooke et al., Biorganic & Medicinal Chemistry 22 (2014) 1029-1039; T. V. Pyrkov et al., ChemMedChem 2013, 8, 1322-1329).

Many different cancer types exhibit an overexpression of PFK-2, particularly its isozymes PFKFB4 and hypoxia-inducible form PFKFB3. PFKFB3 is overexpressed in many cancer types including colon, prostate, pancreatic, breast, thyroid, leukemia, lung, ovarian tumors (D. G. Brooke et al., Biorganic & Medicinal Chemistry 22 (2014) 1029-1039; T. V. Pyrkov et al., ChemMedChem 2013, 8, 1322-1329). Overexpression of PFKFB4 has been associated, inter alia, with glioma, hepatic, bladder, and prostate cancer (T. V. Pyrkov et al., ChemMedChem 2013, 8, 1322-1329). Thus, 6-phosphofructo-2-kinase/fructose-2,6-bisphosphatase and in particular isoforms PFKFB3 and PFKFB4 are promising targets for cancer therapy by utilizing small molecules as inhibitors of these enzymes.

International Patent Application PCT/EP2016/000783 (filed on May 12, 2016, published as WO 2016/180536 A1) describes certain substituted quinoxaline derivatives as inhibitors of PFKFB3 and/or PFKFB4 that inhibitors being be useful for the prevention and/or treatment of medical conditions, disorders and/or diseases that are affected by PFKFB3 and/or PFKFB4 activity, in particular cancer diseases.

### DESCRIPTION OF THE INVENTION

However, there is still a need to further improve those PFKFB3 and/or PFKFB4 inhibitors described in that International Patent Application PCT/EP2016/000783 (published as WO 2016/180536 A1) with regard to activity and/or solubility and/or metabolic stability.

It has been surprisingly found that the compounds of the present invention of formula (I) as defined below are active PFKFB3 and/or PFKFB4 inhibitors:

Thus, one subject of the present invention is a compound of formula (I)

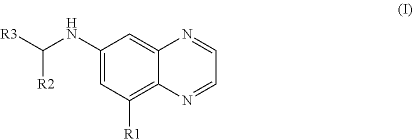

wherein
R1 denotes N-methyl-indol-6-yl(1-methyl-1H-indol-6-yl), 3-methyl-1-benzofuran-5-yl, 1-methyl-1H-pyrrolo[3,2-b]pyridin-6-yl;
R2 denotes 1H-pyrazol-4-yl or 1-methyl-1H-pyrazol-4-yl and
R3 denotes 1H-imidazol-2-yl, 1-methyl-1H-imidazol-2-yl, 1H-imidazol-5-yl, 1-methyl-1H-imidazol-5-yl, 1H-1,2,3-triazol-5-yl, 1-methyl-1H-1,2,3-triazol-5-yl, morpholin-2-yl, morpholin-3-yl, pyridin-3-yl, pyridin-4-yl, 4H-1,2,4-triazol-3-yl, 4-methyl-4H-1,2,4-triazol-3-yl;
or
R2 denotes 1H-pyrazol-3-yl or 1-methyl-1H-pyrazol-3-yl and
R3 denotes 1H-1,2,3-triazol-5-yl (=3H-1,2,3-triazol-4-yl), 1-methyl-1H-1,2,3-triazol-5-yl (=3-methyl-3H-1,2,3-triazol-4-yl), 4H-1,2,4-triazol-3-yl, 4-methyl-4H-1,2,4-triazol-3-yl;
or
R2 denotes 1H-pyridazin-6-on-3-yl, 6-methoxypyridazin-3-yl and
R3 denotes pyridin-3-yl, pyridin-4-yl;
or derivatives, N-oxides, prodrugs, solvates, tautomers or stereoisomers thereof as well as the physiologically acceptable salts of each of the foregoing, including mixtures thereof in all ratios.

Any of those preferred or particular embodiments of the present invention as specified below and in the claims do not only refer to the specified compounds of formula (I) but to derivatives, N-oxides, prodrugs, solvates, tautomers or stereoisomers thereof as well as the physiologically acceptable salts of each of the foregoing, including mixtures thereof in all ratios, too, unless indicated otherwise.

In a particular embodiment, PE1, the compound of the present invention is a compound of formula (I) wherein
R1 denotes N-methyl-indol-6-yl(1-methyl-1H-indol-6-yl), 3-methyl-1-benzofuran-5-yl;
R2 denotes 1-methyl-1H-pyrazol-4-yl and
R3 denotes 1-methyl-1H-imidazol-2-yl, 1-methyl-1H-imidazol-5-yl, 1-methyl-1H-1,2,3-triazol-5-yl, morpholin-2-yl, pyridin-3-yl, 4-methyl-4H-1,2,4-triazol-3-yl;

In a preferred embodiment, PE1a, of this particular embodiment PE1, a compound of the present invention is a compound of formula (I) wherein
R1 denotes N-methyl-indol-6-yl(1-methyl-1H-indol-6-yl), 3-methyl-1-benzofuran-5-yl;
R2 denotes 1-methyl-1H-pyrazol-4-yl and
R3 denotes 1-methyl-1H-imidazol-2-yl, 1-methyl-1H-imidazol-5-yl, 1-methyl-1H-1,2,3-triazol-5-yl, morpholin-2-yl, pyridin-3-yl, 4-methyl-4H-1,2,4-triazol-3-yl.

It is even more preferred that for a compound of embodiment PE1a R3 is selected from one of the 5-membered heterocycles, in particular 1-methyl-1H-imidazol-2-yl, 1-methyl-1H-imidazol-5-yl, 1-methyl-1H-1,2,3-triazol-5-yl, especially 1-methyl-1H-1,2,3-triazol-5-yl.

In another particular embodiment, PE2, the compound of the present invention is a compound of formula (I) wherein
R1 denotes N-methyl-indol-6-yl;
R2 denotes 1-methyl-1H-pyrazol-3-yl and
R3 denotes 1-methyl-1H-1,2,3-triazol-5-yl, 4-methyl-4H-1,2,4-triazol-3-yl.

In still another particular embodiment, PE3, the compound of the present invention is a compound of formula (I) wherein
R1 denotes N-methyl-indol-6-yl;
R2 denotes 1H-pyridazin-6-on-3-yl or 6-methoxypyridazin-3-yl and
R3 denotes pyridine-3-yl.

In yet another particular embodiment, PE4, a compound of the present invention is a compound selected from the following group of compounds, or an N-oxide thereof and/or a pharmaceutically acceptable salt thereof, the group consisting of:
6-[{[8-(1-methyl-1H-indol-6-yl)quinoxalin-6-yl]amino}(pyridin-3-yl)methyl]-2,3-dihydropyridazin-3-one (6-{[8-(1-Methyl-1H-indol-6-yl)-quinoxalin-6-ylamino]-pyridin-3-yl-methyl}-2H-pyridazin-3-one)
6-[(S)-{[8-(1-methyl-1H-indol-6-yl)quinoxalin-6-yl]amino}(pyridin-3-yl)methyl]-2,3-dihydropyridazin-3-one
6-[(R)-{[8-(1-methyl-1H-indol-6-yl)quinoxalin-6-yl]amino}(pyridin-3-yl)methyl]-2,3-dihydropyridazin-3-one
N-[(1-methyl-1H-imidazol-2-yl)(1-methyl-1H-pyrazol-4-yl)methyl]-8-(1-methyl-1H-indol-6-yl)quinoxalin-6-amine
N—[(S)-(1-methyl-1H-imidazol-2-yl)(1-methyl-1H-pyrazol-4-yl)methyl]-8-(1-methyl-1H-indol-6-yl)quinoxalin-6-amine
N—[(R)-(1-methyl-1H-imidazol-2-yl)(1-methyl-1H-pyrazol-4-yl)methyl]-8-(1-methyl-1H-indol-6-yl)quinoxalin-6-amine
N-[(6-methoxypyridazin-3-yl)(pyridin-3-yl)methyl]-8-(1-methyl-1H-indol-6-yl)quinoxalin-6-amine
N—[(S)-(6-methoxypyridazin-3-yl)(pyridin-3-yl)methyl]-8-(1-methyl-1H-indol-6-yl)quinoxalin-6-amine
N—[(R)-(6-methoxypyridazin-3-yl)(pyridin-3-yl)methyl]-8-(1-methyl-1H-indol-6-yl)quinoxalin-6-amine
N-[(1-methyl-1H-1,2,3-triazol-5-yl)(1-methyl-1H-pyrazol-4-yl)methyl]-8-(1-methyl-1H-indol-6-yl)quinoxalin-6-amine ([8-(1-Methyl-1H-indol-6-yl)-quinoxalin-6-yl]-[(1-methyl-1H-pyrazol-4-yl)-(3-methyl-3H-[1,2,3]triazol-4-yl)-methyl]-amine)
N—[(S)-(1-methyl-1H-1,2,3-triazol-5-yl)(1-methyl-1H-pyrazol-4-yl)methyl]-8-(1-methyl-1H-indol-6-yl)quinoxalin-6-amine
[8-(1-Methyl-1H-indol-6-yl)-quinoxalin-6-yl]-[(S)-(1-methyl-1H-pyrazol-4-yl)-(3-methyl-3H-[1,2,3]triazol-4-yl)-methyl]-amine
N—[(R)-(1-methyl-1H-1,2,3-triazol-5-yl)(1-methyl-1H-pyrazol-4-yl)methyl]-8-(1-methyl-1H-indol-6-yl)quinoxalin-6-amine
[8-(1-Methyl-1H-indol-6-yl)-quinoxalin-6-yl]-[(R)-(1-methyl-1H-pyrazol-4-yl)-(3-methyl-3H-[1,2,3]triazol-4-yl)-methyl]-amine
N-(1-methyl-1H-imidazol-5-yl)(1-methyl-1H-pyrazol-4-yl)methyl]-8-(1-methyl-1H-indol-6-yl)quinoxalin-6-amine ([(3-Methyl-3H-imidazol-4-yl)-(1-methyl-1H-pyrazol-4-yl)-methyl]-[8-(1-methyl-1H-indol-6-yl)-quinoxalin-6-yl]-amine)
[(3-Methyl-3H-imidazol-4-yl)-(1-methyl-1H-pyrazol-4-yl)-methyl]-[8-(1-methyl-1H-indol-6-yl)-quinoxalin-6-yl]-amine
N—[(S)-(1-methyl-1H-imidazol-5-yl)(1-methyl-1H-pyrazol-4-yl)methyl]-8-(1-methyl-1H-indol-6-yl)quinoxalin-6-amine
[(S)-(3-Methyl-3H-imidazol-4-yl)-(1-methyl-1H-pyrazol-4-yl)-methyl]-[8-(1-methyl-1H-indol-6-yl)-quinoxalin-6-yl]-amine
N—[(R)-(1-methyl-1H-imidazol-5-yl)(1-methyl-1H-pyrazol-4-yl)methyl]-8-(1-methyl-1H-indol-6-yl)quinoxalin-6-amine
[(R)-(3-Methyl-3H-imidazol-4-yl)-(1-methyl-1H-pyrazol-4-yl)-methyl]-[8-(1-methyl-1H-indol-6-yl)-quinoxalin-6-yl]-amine
N-[(1-methyl-1H-1,2,3-triazol-5-yl)(1-methyl-1H-pyrazol-3-yl)methyl]-8-(1-methyl-1H-indol-6-yl)quinoxalin-6-amine([8-(1-Methyl-1H-indol-6-yl)-quinoxalin-6-yl]-[(1-methyl-1H-pyrazol-3-yl)-(3-methyl-3H-[1,2,3]triazol-4-yl)-methyl]-amine)
N—[(S)-(1-methyl-1H-1,2,3-triazol-5-yl)(1-methyl-1H-pyrazol-3-yl)methyl]-8-(1-methyl-1H-indol-6-yl)quinoxalin-6-amine
N—[(R)-(1-methyl-1H-1,2,3-triazol-5-yl)(1-methyl-1H-pyrazol-3-yl)methyl]-8-(1-methyl-1H-indol-6-yl)quinoxalin-6-amine
8-(1-methyl-1H-indol-6-yl)-N-[(1-methyl-1H-pyrazol-4-yl)(morpholin-2-yl)methyl]quinoxalin-6-amine
8-(1-methyl-1H-indol-6-yl)-N—[(S)-(1-methyl-1H-pyrazol-4-yl)(morpholin-2-yl)methyl] quinoxalin-6-amine
8-(1-methyl-1H-indol-6-yl)-N—[(R)-(1-methyl-1H-pyrazol-4-yl)(morpholin-2-yl)methyl]]quinoxalin-6-amine
8-(1-methyl-1H-indol-6-yl)-N-[(1-methyl-1H-pyrazol-4-yl)(pyridin-3-yl)methyl]quinoxalin-6-amine
8-(1-methyl-1H-indol-6-yl)-N—[(R)-(1-methyl-1H-pyrazol-4-yl)(pyridin-3-yl)methyl]quinoxalin-6-amine
8-(1-methyl-1H-indol-6-yl)-N—[(S)-(1-methyl-1H-pyrazol-4-yl)(pyridin-3-yl)methyl]quinoxalin-6-amine
8-(1-methyl-1H-indol-6-yl)-N-[(1-methyl-1H-pyrazol-4-yl)(4-methyl-4H-1,2,4-triazol-3-yl)methyl]quinoxalin-6-amine
8-(1-methyl-1H-indol-6-yl)-N—[(S)-(1-methyl-1H-pyrazol-4-yl)(4-methyl-4H-1,2,4-triazol-3-yl)methyl]quinoxalin-6-amine
8-(1-methyl-1H-indol-6-yl)-N—[(S)-(1-methyl-1H-pyrazol-4-yl)(4-methyl-4H-1,2,4-triazol-3-yl)methyl]quinoxalin-6-amine
8-(1-methyl-1H-indol-6-yl)-N-[(1-methyl-1H-pyrazol-3-yl)(4-methyl-4H-1,2,4-triazol-3-yl)methyl]quinoxalin-6-amine
8-(1-methyl-1H-indol-6-yl)-N—[(S)-(1-methyl-1H-pyrazol-3-yl)(4-methyl-4H-1,2,4-triazol-3-yl)methyl]quinoxalin-6-amine
8-(1-methyl-1H-indol-6-yl)-N—[(S)-(1-methyl-1H-pyrazol-3-yl)(4-methyl-4H-1,2,4-triazol-3-yl)methyl]quinoxalin-6-amine
8-(3-methyl-1-benzofuran-5-yl)-N-[(1-methyl-1H-1,2,3-triazol-5-yl)(1-methyl-1H-pyrazol-4-yl)methyl]quinoxalin-6-amine ([8-(3-Methyl-benzofuran-5-yl)-quinoxalin-6-yl]-[(1-methyl-1H-pyrazol-4-yl)-(3-methyl-3H-[1,2,3]triazol-4-yl)-methyl]-amine)
[8-(3-Methyl-benzofuran-5-yl)-quinoxalin-6-yl]-[(1-methyl-1H-pyrazol-4-yl)-(3-methyl-3H-[1,2,3]triazol-4-yl)-methyl]-amine
8-(3-methyl-1-benzofuran-5-yl)-N—[(S)-(1-methyl-1H-1,2,3-triazol-5-yl)(1-methyl-1H-pyrazol-4-yl)methyl]quinoxalin-6-amine
[8-(3-Methyl-benzofuran-5-yl)-quinoxalin-6-yl]-[(S)-(1-methyl-1H-pyrazol-4-yl)-(3-methyl-3H-[1,2,3]triazol-4-yl)-methyl]-amine
8-(3-methyl-1-benzofuran-5-yl)-N—[(S)-(1-methyl-1H-1,2,3-triazol-5-yl)(1-methyl-1H-pyrazol-4-yl)methyl]quinoxalin-6-amine
[8-(3-Methyl-benzofuran-5-yl)-quinoxalin-6-yl]-[(R)-(1-methyl-1H-pyrazol-4-yl)-(3-methyl-3H-[1,2,3]triazol-4-yl)-methyl]-amine
[8-(3-Methyl-benzofuran-5-yl)-quinoxalin-6-yl]-[(1-methyl-1H-pyrazol-4-yl)-(3-methyl-3H-[1,2,3]triazol-4-yl)-methyl]-amine
[8-(3-Methyl-benzofuran-5-yl)-quinoxalin-6-yl]-[(S)-(1-methyl-1H-pyrazol-4-yl)-(3-methyl-3H-[1,2,3]triazol-4-yl)-methyl]-amine
[8-(3-Methyl-benzofuran-5-yl)-quinoxalin-6-yl]-[(R)-(1-methyl-1H-pyrazol-4-yl)-(3-methyl-3H-[1,2,3]triazol-4-yl)-methyl]-amine; as well as
N-[(6-methoxypyridin-3-yl)(morpholin-2-yl)methyl]-8-(1-methyl-1H-indol-6-yl)quinoxalin-6-amine
N—[(S)-(6-methoxypyridin-3-yl)(morpholin-2-yl)methyl]-8-(1-methyl-1H-indol-6-yl)quinoxalin-6-amine
N—[(R)-(6-methoxypyridin-3-yl)(morpholin-2-yl)methyl]-8-(1-methyl-1H-indol-6-yl)quinoxalin-6-amine
[8-(1-Methyl-1H-indol-6-yl)-quinoxalin-6-yl]-(2-methyl-1-pyridin-3-yl-propyl)-amine
[8-(1-Methyl-1H-indol-6-yl)-quinoxalin-6-yl]-((R)-2-methyl-1-pyridin-3-yl-propyl)-amine
[8-(1-Methyl-1H-indol-6-yl)-quinoxalin-6-yl]-((S)-2-methyl-1-pyridin-3-yl-propyl)-amine
N-[2-(1-Methyl-1H-1,2,3-triazol-5-yl)propan-2-yl]-8-(1-methyl-1H-indol-6-yl)quinoxalin-6-amine
8-(1-Methyl-1H-indol-6-yl)-N-[2-(morpholin-2-yl)propan-2-yl]quinoxalin-6-amine
[(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine
[(S)-(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine
[(R)-(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine.

In a preferred embodiment, PE4a, of particular embodiment PE4 the compound is selected from the group of compounds, or their respective N-oxides and/or a pharmaceutically acceptable salt thereof, that is consisting of:
6-[{[8-(1-methyl-1H-indol-6-yl)quinoxalin-6-yl]amino}(pyridin-3-yl)methyl]-2,3-dihydropyridazin-3-one
6-[(S)-{[8-(1-methyl-1H-indol-6-yl)quinoxalin-6-yl]amino}(pyridin-3-yl)methyl]-2,3-dihydropyridazin-3-one
6-[(R)-{[8-(1-methyl-1H-indol-6-yl)quinoxalin-6-yl]amino}(pyridin-3-yl)methyl]-2,3-dihydropyridazin-3-one
N—[(S)-(1-methyl-1H-imidazol-2-yl)(1-methyl-1H-pyrazol-4-yl)methyl]-8-(1-methyl-1H-indol-6-yl)quinoxalin-6-amine
N—[(R)-(1-methyl-1H-imidazol-2-yl)(1-methyl-1H-pyrazol-4-yl)methyl]-8-(1-methyl-1H-indol-6-yl)quinoxalin-6-amine
N—[(S)-(6-methoxypyridazin-3-yl)(pyridin-3-yl)methyl]-8-(1-methyl-1H-indol-6-yl)quinoxalin-6-amine
N—[(R)-(6-methoxypyridazin-3-yl)(pyridin-3-yl)methyl]-8-(1-methyl-1H-indol-6-yl)quinoxalin-6-amine
N—[(S)-(1-methyl-1H-1,2,3-triazol-5-yl)(1-methyl-1H-pyrazol-4-yl)methyl]-8-(1-methyl-1H-indol-6-yl)quinoxalin-6-amine
N—[(R)-(1-methyl-1H-1,2,3-triazol-5-yl)(1-methyl-1H-pyrazol-4-yl)methyl]-8-(1-methyl-1H-indol-6-yl)quinoxalin-6-amine
N—[(S)-(1-methyl-1H-imidazol-5-yl)(1-methyl-1H-pyrazol-4-yl)methyl]-8-(1-methyl-1H-indol-6-yl)quinoxalin-6-amine
N—[(R)-(1-methyl-1H-imidazol-5-yl)(1-methyl-1H-pyrazol-4-yl)methyl]-8-(1-methyl-1H-indol-6-yl)quinoxalin-6-amine
8-(1-methyl-1H-indol-6-yl)-N—[(R)-(1-methyl-1H-pyrazol-4-yl)(pyridin-3-yl)methyl]quinoxalin-6-amine
8-(1-methyl-1H-indol-6-yl)-N—[(S)-(1-methyl-1H-pyrazol-4-yl)(pyridin-3-yl)methyl]quinoxalin-6-amine
8-(3-methyl-1-benzofuran-5-yl)-N—[(S)-(1-methyl-1H-1,2,3-triazol-5-yl)(1-methyl-1H-pyrazol-4-yl)methyl]quinoxalin-6-amine
8-(3-methyl-1-benzofuran-5-yl)-N—[(S)-(1-methyl-1H-1,2,3-triazol-5-yl)(1-methyl-1H-pyrazol-4-yl)methyl]quinoxalin-6-amine
N—[(S)-(1-methyl-1H-1,2,3-triazol-5-yl)(1-methyl-1H-pyrazol-3-yl)methyl]-8-(1-methyl-1H-indol-6-yl)quinoxalin-6-amine
N—[(R)-(1-methyl-1H-1,2,3-triazol-5-yl)(1-methyl-1H-pyrazol-3-yl)methyl]-8-(1-methyl-1H-indol-6-yl)quinoxalin-6-amine.

As will be recognized by the person skilled in the art of organic and medicinal chemistry that the compounds of formula (I) and hence of all the particular and preferred embodiments described herein have at least on centre of chirality which is indicated in the formula (I*) below by using an asterisk (*):

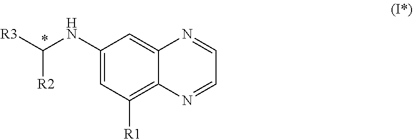

This centre of chirality is the carbon atom adjacent to the —NH— moiety attached to the quinoxaline core of the compound of formula (I) which carbon atom is further substituted by three different substituents, R2, R3 and a hydrogen atom. The compounds of formula (I) may have further centres of chirality. They may accordingly occur in various enantiomeric and diastereomeric forms, as the case may be, and be in racemic or optically active form. The invention, therefore, also relates to the optically active forms, enantiomers, racemates, diastereomers, mixtures thereof in all ratios, collectively: “stereoisomers” for the purpose of the present invention, of these compounds. Since the pharmaceutical activity of the racemates or stereoisomers of the compounds according to the invention may differ, it may be desirable to use a specific stereoisomer, e.g. one specific enantiomer or diastereomer. In these cases, a compound according to the present invention obtained as a racemate—or even intermediates thereof—may be separated into the stereoisomeric (enantiomeric, diastereoisomeric) compounds by chemical or physical measures known to the person skilled in the art. Another approach that may be applied to obtain one or more specific stereoisomers of a compound of the present invention in an enriched or pure form makes use of stereoselective synthetic procedures, e.g. applying starting material in a stereoisomerically enriched or pure form (for instance using the pure or enriched (R)- or (S)-enantiomer of a particular starting material bearing a chiral center) or utilizing chiral reagents or catalysts, in particular enzymes. In the context of the present invention the term “pure enantiomer” usually refers to a relative purity of one enantiomer over the other (its antipode) of equal to or greater than 95%, preferably ≥98%, more preferably ≥98.5%, still more preferably ≥99%.

Thus, for example, the compounds of the invention which have one or more centers of chirality and which occur as racemates or as mixtures of enantiomers or diastereoisomers can be fractionated or resolved by methods known per se into their optically pure or enriched isomers, i.e. enantiomers or diastereomers. The separation of the compounds of the invention can take place by chromatographic methods, e.g. column separation on chiral or nonchiral phases, or by recrystallization from an optionally optically active solvent or by use of an optically active acid or base or by derivatization with an optically active reagent such as, for example, an optically active alcohol, and subsequent elimination of the radical.

Those compounds of the present invention having one and only one centre of chirality, i.e. compounds of formula (I*), are preferred embodiments, PE5, of the present invention. In a particularly preferred embodiment, PE5a, the compound of the present invention is selected to be only one of the two enantiomers, either the (S)- or the (R)-enantiomer, most preferably the enantiomer exhibiting the higher inhibitory activity on PFKFB3 and/or PFKFB4, in particular PFKFB3. It is to be noted that the compounds of the present invention having only one chiral centre have been prepared not only in racemic form, i.e. as a 1:1 mixture of both enantiomers, but in pure enantiomeric form as well; both preparation and identification of all enantiomers are reproducible. However, ascribing the absolute configuration to each enantiomeric compound of the present invention with absolute certainty may not have been achieved yet. Thus, while it is possible to prepare, identify and describe with certainty both the more active and the less active enantiomer, i.e. to establish their relative stereochemistry, the designation of the absolute configuration, i.e. (S)- or (R)-configuration may have to be adapted once it is established by appropriate means, e.g., x-ray structure analysis.

It is an even more preferred embodiment, PE6, that the compound of the present invention is one of the two optical isomers, i.e. enantiomers of N-[(1-methyl-1H-1,2,3-triazol-5-yl)(1-methyl-1H-pyrazol-4-yl)methyl]-8-(1-methyl-1H-indol-6-yl)quinoxalin-6-amine, most preferably that enantiomer with the higher inhibitory activity on PFKFB3. While it is currently believed that this more active stereoisomer happens to be the S-enantiomer, N—[(S)-(1-methyl-1H-1,2,3-triazol-5-yl)(1-methyl-1H-pyrazol-4-yl)methyl]-8-(1-methyl-1H-indol-6-yl)quinoxalin-6-amine, it may well be that the higher inhibitory activity on PFKFB3 has to be ascribed to the opposite enantiomer, N—[(R)-(1-methyl-1H-1,2,3-triazol-5-yl)(1-methyl-1H-pyrazol-4-yl)methyl]-8-(1-methyl-1H-indol-6-yl)quinoxalin-6-amine, once the absolute configuration of the compound having the higher activity has been established by appropriate means, for instance, of x-ray structure analysis.

The compounds of the invention are structurally similar to those quinoxaline derivatives described in the International Patent Application PCT/EP2016/000783 (filed on May 12, 2016, published as WO 2016/180536 A1). However, a compound of the present invention may show at least one property being improved in comparison to a similar quinoxaline derivative described in PCT/EP2016/000783 (published as WO 2016/180536 A1), that property may be any chemical, physical, physicochemical and/or pharmacological property that may have an effect on the safe and/or effective use of the compound as a pharmaceutical active or medicament. That property may be selected (without limitation) from inhibitory activity against PFKFB3 and/or PFKFB4, in particular PFKFB3, selectivity, solubility (kinetic solubility, thermodynamic solubility), metabolic or microsomal stability, diminished undesired effects, and the like.

As used herein, the following definitions shall apply unless otherwise indicated or defined specifically elsewhere in the description and/or the claims for specific substituents, radicals, groups, moieties or terms.

“Hal” denotes F, Cl, Br or I, in particular Cl, Br or I.

In the context of the present invention the term “derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

The compounds of the present invention can be in the form of a prodrug compound. “Prodrug” and “prodrug compound” mean a derivative that is converted into a biologically active compound according to the present invention under physiological conditions in the living body, e.g., by oxidation, reduction, hydrolysis or the like, each of which is carried out enzymatically, or without enzyme involvement. Examples of prodrugs are compounds, in which the amino group in a compound of the present invention is acylated, alkylated or phosphorylated, e.g., eicosanoylamino, alanylamino, pivaloyloxymethylamino or in which the hydroxyl group is acylated, alkylated, phosphorylated or converted into the borate, e.g. acetyloxy, palmitoyloxy, pivaloyloxy, succinyloxy, fumaryloxy, alanyloxy or in which the carboxyl group is esterified or amidated, or in which a sulfhydryl group forms a disulfide bridge with a carrier molecule, e.g. a peptide, that delivers the drug selectively to a target and/or to the cytosol of a cell. These compounds can be produced from compounds of the present invention according to well-known methods. Other examples of prodrugs are compounds, wherein the carboxylate in a compound of the present invention is for example converted into an alkyl-, aryl-, choline-, amino-, acyloxymethylester, linolenoyl-ester.

The term “solvates” means addition forms of the compounds of the present invention with solvents, preferably pharmaceutically acceptable solvents, that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, e.g. a mono- or dihydrate. If the solvent is alcohol, the solvate formed is an alcoholate, e.g., a methanolate or ethanolate. If the solvent is an ether, the solvate formed is an etherate, e.g., diethyl etherate.

The term “N-oxides” means such compounds of the present invention that contain an amine oxide moiety, i.e. the oxide of a tertiary amine group.

In the context of the present invention the term “tautomer” refers to compounds of the present invention that may exist in tautomeric forms and show tautomerism; for instance, carbonyl compounds may be present in their keto and/or their enol form and show keto-enol tautomerism. Those tautomers may occur in their individual forms, e.g., the keto or the enol form, or as mixtures thereof and are claimed separately and together as mixtures in any ratio. The same applies for cis/trans isomers, E/Z isomers, conformers and the like.

The compounds of the present invention can be in the form of a pharmaceutically acceptable salt, a pharmaceutically acceptable solvate, or a pharmaceutically acceptable solvate of a pharmaceutically acceptable salt.

The term “pharmaceutically acceptable salts” refers to salts prepared from pharmaceutically acceptable bases or acids, including inorganic bases or acids and organic bases or acids. In cases where the compounds of the present invention contain one or more acidic or basic groups, the invention also comprises their corresponding pharmaceutically acceptable salts. Thus, the compounds of the present invention which contain acidic groups can be present in salt form, and can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the present invention which contain one or more basic groups, e.g. groups which can be protonated, can be present in salt form, and can be used according to the invention in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, malonic acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid or aspartic acid, and other acids known to the person skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates and glutamates. The stoichiometry of the salts formed from the compounds of the invention may moreover be an integral or non-integral multiple of one.

If the compounds of the present invention simultaneously contain acidic and basic groups in the molecule, the invention also includes, in addition to the salt forms mentioned, inner salts or betaines (zwitterions). The respective salts can be obtained by customary methods which are known to a person skilled in the art, for example by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Therefore, the following items are also in accordance with the invention:
(a) all stereoisomers or tautomers of the compounds, including mixtures thereof in all ratios;
(b) prodrugs of the compounds, or stereoisomers or tautomers of these prodrugs;
(c) pharmaceutically acceptable salts of the compounds and of the items mentioned under (a) and (b);
(d) pharmaceutically acceptable solvates of the compounds and of the items mentioned under (a), (b) and (c);
(e) N-oxides of the compounds and of the items mentioned under (a), (b), (c), and (d).

It should be understood that all references to compounds above and below are meant to include these items, in particular pharmaceutically acceptable solvates of the compounds, or pharmaceutically acceptable solvates of their pharmaceutically acceptable salts.

Furthermore, the present invention relates to pharmaceutical compositions comprising at least one compound of formula (I), or its derivatives, prodrugs, solvates, tautomers or stereoisomers thereof as well as the physiologically acceptable salts of each of the foregoing, including mixtures thereof in all ratios, as active ingredient, together with a pharmaceutically acceptable carrier.

For the purpose of the present invention the term “pharmaceutical composition” refers to a composition or product comprising one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier. It may further comprise physiologically acceptable excipients, auxiliaries, adjuvants, diluents and/or additional pharmaceutically active substance other than the compounds of the invention.

The pharmaceutical compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

A pharmaceutical composition of the present invention may additionally comprise one or more other compounds as active ingredients (drugs), such as one or more additional compounds of the present invention. In a particular embodiment the pharmaceutical composition further comprises a second active ingredient or its derivatives, prodrugs, solvates, tautomers or stereoisomers thereof as well as the physiologically acceptable salts of each of the foregoing, including mixtures thereof in all ratios, wherein that second active ingredient is other than a compound of formula (I); preferably, that second active ingredient is a compound that is useful in the treatment, prevention, suppression and/or amelioration of medicinal conditions or pathologies for which the compounds of the present invention are useful as well and which are listed elsewhere hereinbefore or hereinafter. Such combination of two or more active ingredients or drugs may be safer or more effective than either drug or active ingredient alone, or the combination is safer or more effective than it would be expected based on the additive properties of the individual drugs. Such other drug(s) may be administered, by a route and in an amount commonly used contemporaneously or sequentially with a compound of the invention. When a compound of the invention is used contemporaneously with one or more other drugs or active ingredients, a combination product containing such other drug(s) and the compound of the invention—also referred to as “fixed dose combination”—is preferred. However, combination therapy also includes therapies in which the compound of the present invention and one or more other drugs are administered on different overlapping schedules. It is contemplated that when used in combination with other active ingredients, the compound of the present invention or the other active ingredient or both may be used effectively in lower doses than when each is used alone. Accordingly, the pharmaceutical compositions of the present invention include those that contain one or more other active ingredients, in addition to a compound of the invention.

The compounds of the present invention can be used as medicaments. They exhibit pharmacological activity by inhibiting 6-phosphofructo-2-kinase/fructose-2,6-bisphosphatase (PFKFB), in particular its isoforms PFKFB3 and/or PFKFB4, more particular PFKFB3. Even more particular, the compounds of the present invention exhibit inhibition of the kinase enzymatic activity of PFKFB, especially of PFKFB3 and/or PFKFB4, more especially of PFKFB3. Thus, they are useful for the treatment, prevention, suppression and/or amelioration of medicinal conditions or pathologies that are affected by PFKFB activity, in particular by PFKFB3 and/or PFKFB4 activity, more particular by PFKFB3 activity. The compounds of the present invention are thus particularly useful for the treatment of a hyperproliferative disorder. More specifically, they are useful for the treatment of a disorder or disease selected from the group consisting of cancer, in particular adipose cancer, anogenital cancer, bladder cancer, breast cancer, central nervous system cancer, cervical cancer, colon cancer, connective tissue cancer, glioblastoma, glioma, kidney cancer, leukemia, lung cancer, lymphoid cancer, ovarian cancer, pancreatic cancer, prostate cancer, retinal cancer, skin cancer, stomach cancer, uterine cancer.

The disclosed compounds of the formula (I) can be administered and/or used in combination with other known therapeutic agents, including anticancer agents. As used herein, the term “anticancer agent” relates to any agent which is administered to a patient with cancer for the purposes of treating the cancer.

The anti-cancer treatment defined above may be applied as a monotherapy or may involve, in addition to the herein disclosed compounds of formula (I), conventional surgery or radiotherapy or medicinal therapy. Such medicinal therapy, e.g. a chemotherapy or a targeted therapy, may include one or more, but preferably one, of the following anti-tumor agents:

### Alkylating Agents

such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-302⁴, VAL-083⁴;

### Platinum Compounds

such as carboplatin, cisplatin, eptaplatin, miriplatine hydrate, oxaliplatin, lobaplatin, nedaplatin, picoplatin, satraplatin;

### DNA Altering Agents

such as amrubicin, bisantrene, decitabine, mitoxantrone, procarbazine, trabectedin, clofarabine;
amsacrine, brostallicin, pixantrone, laromustine^{1,3};

### Topoisomerase Inhibitors

such as etoposide, irinotecan, razoxane, sobuzoxane, teniposide, topotecan; amonafide, belotecan, elliptinium acetate, voreloxin;

### Microtubule Modifiers

such as cabazitaxel, docetaxel, eribulin, ixabepilone, paclitaxel, vinblastine, vincristine, vinorelbine, vindesine, vinflunine;
fosbretabulin, tesetaxel;

### Antimetabolites

such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur;
doxifluridine, elacytarabine, raltitrexed, sapacitabine, tegafur^{2,3}, trimetrexate;

### Anticancer Antibiotics

such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin;
aclarubicin, peplomycin, pirarubicin;

### Hormones/Antagonists

such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol;
acolbifene, danazol, deslorelin, epitiostanol, orteronel, enzalutamide^{1,3};

### Aromatase Inhibitors

such as aminoglutethimide, anastrozole, exemestane, fadrozole, letrozole, testolactone;
formestane;

### Small Molecule Kinase Inhibitors

such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib;
afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib⁴, cabozantinib S-malate^{1,3}, ibrutinib^{1,3}, icotinib⁴, buparlisib², cipatinib⁴, cobimetinib^{1,3}, idelalisib^{1,3}, fedratinibl, XL-647⁴;

### Photosensitizers

such as methoxsalen³;
porfimer sodium, talaporfin, temoporfin;

### Antibodies

such as alemtuzumab, besilesomab, brentuximab vedotin, cetuximab, denosumab, ipilimumab, ofatumumab, panitumumab, rituximab, tositumomab, trastuzumab, bevacizumab, pertuzumab^{2,3};
catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomabl, tabalumab^{1,3}, EMD-525797⁴, nivolumab^{1,3};

### Cytokines

such as aldesleukin, interferon alfa², interferon alfa2a³, interferon alfa2b^{2,3}; celmoleukin, tasonermin, teceleukin, oprelvekin^{1,3}, recombinant interferon beta-1a⁴;

### Drug Conjugates

such as denileukin diftitox, ibritumomab tiuxetan, iobenguane I123, prednimustine, trastuzumab emtansine, estramustine, gemtuzumab, ozogamicin, aflibercept;
cintredekin besudotox, edotreotide, inotuzumab ozogamicin, naptumomab estafenatox, oportuzumab monatox, technetium (99mTc) arcitumomab^{1,3}, vintafolide^{1,3};

### Vaccines

such as sipuleucel³; vitespen³, emepepimut-S³, oncoVAX⁴, rindopepimut³, troVax⁴, MGN-1601⁴, MGN-1703⁴;

### Miscellaneous

alitretinoin, bexarotene, bortezomib, everolimus, ibandronic acid, imiquimod, lenalidomide, lentinan, metirosine, mifamurtide, pamidronic acid, pegaspargase, pentostatin, sipuleucel³, sizofiran, tamibarotene, temsirolimus, thalidomide, tretinoin, vismodegib, zoledronic acid, vorinostat;
celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴, picibanil⁴, reolysin⁴, retaspimycin hydrochloride^{1,3}, trebananib^{2,3}, virulizin⁴, carfilzomib^{1,3}, endostatin⁴, immucothel⁴, belinostat³, MGN-1703⁴; ¹ Prop. INN (Proposed International Nonproprietary Name)² Rec. INN (Recommended International Nonproprietary Names)³ USAN (United States Adopted Name)⁴ no INN.

A further embodiment of the present invention is a process for the manufacture of the pharmaceutical compositions of the present invention, characterized in that one or more compounds according to the invention and one or more compounds selected from the group consisting of solid, liquid or semiliquid excipients, auxiliaries, adjuvants, diluents, carriers and pharmaceutically active agents other than the compounds according to the invention, are converted in a suitable dosage form.

In another aspect of the invention, a set or kit is provided comprising a therapeutically effective amount of at least one compound of the invention and/or at least one pharmaceutical composition as described herein and a therapeutically effective amount of at least one further pharmacologically active substance other than the compounds of the invention. It is preferred that this set or kit comprises separate packs of
a) an effective amount of a compound of formula (I), or its derivatives, prodrugs, solvates, tautomers or stereoisomers thereof as well as the physiologically acceptable salts of each of the foregoing, including mixtures thereof in all ratios, and
b) an effective amount of a further active ingredient that further active ingredient not being a compound of formula (I).

The pharmaceutical compositions of the present invention may be administered by any means that achieve their intended purpose. For example, administration may be via oral, parenteral, topical, enteral, intravenous, intramuscular, inhalant, nasal, intraarticular, intraspinal, transtracheal, transocular, subcutaneous, intraperitoneal, transdermal, or buccal routes. Alternatively, or concurrently, administration may be via the oral route. The dosage administered will be dependent upon the age, health, and weight of the recipient, kind of concurrent treatment, if any, frequency of treatment, and the nature of the effect desired. Parenteral administration is preferred. Oral administration is especially preferred.

Suitable dosage forms include, but are not limited to capsules, tablets, pellets, dragees, semi-solids, powders, granules, suppositories, ointments, creams, lotions, inhalants, injections, cataplasms, gels, tapes, eye drops, solution, syrups, aerosols, suspension, emulsion, which can be produced according to methods known in the art, for example as described below:

Tablets: mixing of active ingredient/s and auxiliaries, compression of said mixture into tablets (direct compression), optionally granulation of part of mixture before compression.

Capsules: mixing of active ingredient/s and auxiliaries to obtain a flowable powder, optionally granulating powder, filling powders/granulate into opened capsules, capping of capsules.

Semi-solids (ointments, gels, creams): dissolving/dispersing active ingredient/s in an aqueous or fatty carrier; subsequent mixing of aqueous/fatty phase with complementary fatty/aqueous phase, homogenization (creams only).

Suppositories (rectal and vaginal): dissolving/dispersing active ingredient/s in carrier material liquified by heat (rectal: carrier material normally a wax; vaginal: carrier normally a heated solution of a gelling agent), casting said mixture into suppository forms, annealing and withdrawal suppositories from the forms.

Aerosols: dispersing/dissolving active agent/s in a propellant, bottling said mixture into an atomizer.

In general, non-chemical routes for the production of pharmaceutical compositions and/or pharmaceutical preparations comprise processing steps on suitable mechanical means known in the art that transfer one or more compounds of the invention into a dosage form suitable for administration to a patient in need of such a treatment. Usually, the transfer of one or more compounds of the invention into such a dosage form comprises the addition of one or more compounds, selected from the group consisting of carriers, excipients, auxiliaries and pharmaceutical active ingredients other than the compounds of the invention. Suitable processing steps include, but are not limited to combining, milling, mixing, granulating, dissolving, dispersing, homogenizing, casting and/or compressing the respective active and non-active ingredients. Mechanical means for performing said processing steps are known in the art, for example from Ullmann's Encyclopedia of Industrial Chemistry, 5th Edition. In this respect, active ingredients are preferably at least one compound of the invention and optionally one or more additional compounds other than the compounds of the invention, which show valuable pharmaceutical properties, preferably those pharmaceutical active agents other than the compounds of the invention, which are disclosed herein.

Particularly suitable for oral use are tablets, pills, coated tablets, capsules, powders, granules, syrups, juices or drops, suitable for rectal use are suppositories, suitable for parenteral use are solutions, preferably oil-based or aqueous solutions, furthermore suspensions, emulsions or implants, and suitable for topical use are ointments, creams or powders. The compounds of the invention may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, dyes, flavours and/or a plurality of further active ingredients, for example one or more vitamins.

Suitable excipients are organic or inorganic substances, which are suitable for enteral (for example oral), parenteral or topical administration and do not react with the compounds of the invention, for example water, vegetable oils, benzyl alcohols, alkylene glycols, polyethylene glycols, glycerol triacetate, gelatine, carbohydrates, such as lactose, sucrose, mannitol, sorbitol or starch (maize starch, wheat starch, rice starch, potato starch), cellulose preparations and/or calcium phosphates, for example tricalcium phosphate or calcium hydrogen phosphate, magnesium stearate, talc, gelatine, tragacanth, methyl cellulose, hydroxypropylmethylcellulose, sodium carboxymethylcellulose, polyvinyl pyrrolidone and/or vaseline.

If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries include, without limitation, flow-regulating agents and lubricants, for example, silica, talc, stearic acid or salts thereof, such as magnesium stearate or calcium stearate, and/or polyethylene glycol. Dragee cores are provided with suitable coatings, which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices or to provide a dosage form affording the advantage of prolonged action, the tablet, dragee or pill can comprise an inner dosage and an outer dosage component the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol, solutions of suitable cellulose preparations such as acetyl-cellulose phthalate, cellulose acetate or hydroxypropylmethyl-cellulose phthalate, are used. Dye stuffs or pigments may be added to the tablets or dragee coatings, for example, for identification or in order to characterize combinations of active compound doses.

Suitable carrier substances are organic or inorganic substances which are suitable for enteral (e.g. oral) or parenteral administration or topical application and do not react with the novel compounds, for example water, vegetable oils, benzyl alcohols, polyethylene glycols, gelatin, carbohydrates such as lactose or starch, magnesium stearate, talc and petroleum jelly. In particular, tablets, coated tablets, capsules, syrups, suspensions, drops or suppositories are used for enteral administration, solutions, preferably oily or aqueous solutions, furthermore suspensions, emulsions or implants, are used for parenteral administration, and ointments, creams or powders are used for topical application. The compounds of the invention can also be lyophilized and the lyophilizates obtained can be used, for example, for the production of injection preparations.

Other pharmaceutical preparations, which can be used orally include push-fit capsules made of gelatine, as well as soft, sealed capsules made of gelatine and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

The liquid forms in which the novel compositions of the present invention may be incorporated for administration orally include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatine.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400).

Aqueous injection suspensions may contain substances, which increase the viscosity of the suspension, including, for example, sodium carboxymethyl cellulose, sorbitol, and/or dextran, optionally, the suspension may also contain stabilizers.

For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronized form, in which case one or more additional physiologically acceptable solvents may be present, for example ethanol. Inhalation solutions can be administered with the aid of conventional inhalers.

Pharmaceutical preparations, which can be used rectally, include, for example, suppositories, which consist of a combination of one or more of the active compounds with a suppository base. Suitable suppository bases are, for example, natural or synthetic triglycerides, or paraffin hydrocarbons. In addition, it is also possible to use gelatine rectal capsules, which consist of a combination of the active compounds with a base. Possible base materials include, for example, liquid triglycerides, polyethylene glycols, or paraffin hydrocarbons.

For use in medicine, the compounds of the present invention may be in the form of pharmaceutically acceptable salts. Other salts may, however, be useful in the preparation of the compounds of the invention or of their pharmaceutically acceptable salts. Suitable pharmaceutically acceptable salts of the compounds of this invention are those described hereinbefore and include acid addition salts which may, for example be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic bases, e.g. quaternary ammonium salts.

The pharmaceutical preparations can be employed as medicaments in human and veterinary medicine. As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Said therapeutic effective amount of one or more of the compounds of the invention is known to the skilled artisan or can be easily determined by standard methods known in the art.

The compounds of the present invention and the optional additional active substances are generally administered analogously to commercial preparations. Usually, suitable doses that are therapeutically effective lie in the range between 0.0005 mg and 1000 mg, preferably between 0.005 mg and 500 mg and especially between 0.5 mg and 100 mg per dose unit. The daily dose is preferably between about 0.001 mg/kg and 10 mg/kg of body weight.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The specific dose for the individual patient, in particular for the individual human patient, depends, however, on the multitude of factors, for example on the efficacy of the specific compounds employed, on the age, body weight, general state of health, the sex, the kind of diet, on the time and route of administration, on the excretion rate, the kind of administration and the dosage form to be administered, the pharmaceutical combination and severity of the particular disorder to which the therapy relates. The specific therapeutic effective dose for the individual patient can readily be determined by routine experimentation, for example by the doctor or physician, which advises or attends the therapeutic treatment.

The compounds of the present invention can be prepared according to the procedures of the following Schemes and Examples, using appropriate materials, and are further exemplified by the following specific examples.

They may also be prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions which are known and suitable for the said reactions. Use can also be made of variants which are known per se, but are not mentioned here in greater detail.

Likewise, the starting materials for the preparation of compounds of the present invention can be prepared by methods as described in the examples or by methods known per se, as described in the literature of synthetic organic chemistry and known to the skilled person, or can be obtained commercially. The starting materials for the processes claimed and/or utilized may, if desired, also be formed in situ by not isolating them from the reaction mixture, but instead immediately converting them further into the compounds of the invention or intermediate compounds. On the other hand, in general it is possible to carry out the reaction stepwise.

Preferably, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, dimethylformamide (DMF) or N-methyl pyrrolidinone (NMP); nitriles, such as acetonitrile; sulfoxides, such as dimethyl sulfoxide (DMSO); nitro compounds, such as nitromethane or nitrobenzene; esters, such as ethyl acetate, or mixtures of the said solvents or mixtures with water.

The reaction temperature is between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The present invention also refers to a process for manufacturing a compound of the present invention, or derivatives, N-oxides, prodrugs, solvates, tautomers or stereoisomers thereof as well as the physiologically acceptable salts of each of the foregoing, the process being characterized in that

(a) a compound of formula (II)

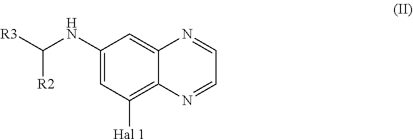

wherein
Hal1 denotes Cl, Br or I;
R2 and R3 have the same meaning as defined above or in claims 1 to 7 for compounds of formula (I);
is reacted under C—C coupling reaction conditions which conditions may utilize one or more suitable C—C coupling reaction reagents including catalysts
with a compound R1-RG1
wherein
R1 has the same meaning as defined above or in claims 1 to 7 for compounds of formula (I);
RG1 denotes a chemical moiety being reactive under the particular C—C coupling reaction conditions utilized;
or
   (b) a compound of formula (III)

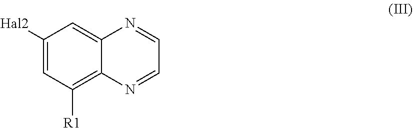

wherein
Hal2 denotes Cl, Br or I;
R1 has the same meaning as defined above or in claims 1 to 7 for compounds of formula (I);
is reacted under C—N coupling reaction conditions which conditions may utilize one or more suitable C—N coupling reaction reagents including catalysts
with a compound R2R3HC-NH-RG2
wherein
R2 and R3 have the same meaning as defined above or in claims 1 to 7 for compounds of formula (I);
RG2 denotes a chemical moiety being reactive under the particular C—N coupling reaction conditions utilized.

A particularly versatile starting point for making compounds of formula (I) are 5-bromo-7-chloroquinoxaline (Int 2) and 7-bromo-5-chloroquinoxaline (Int 3) both of which are readily available by applying in analogy synthetic methods described in WO 2010/20363 A1.

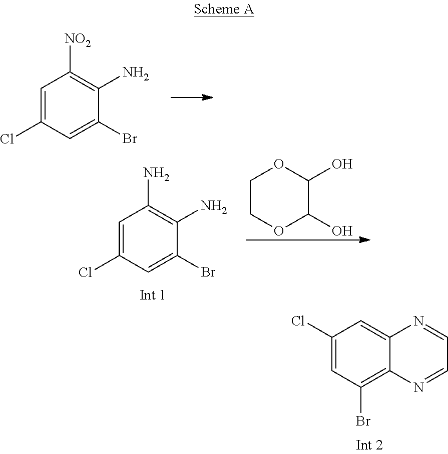

2-Bromo-4-chloro-6-nitrophenylamine is converted into 3-bromo-5-chlorobenzene-1,2-diamine (Int 1) by utilizing suitable reduction means, e.g. tin(II)-chloride, which in turn is converted into 5-bromo-7-chloroquinoxaline (Int 2) by reacting it with 2,3-dihydroxy-1,4-dioxane.

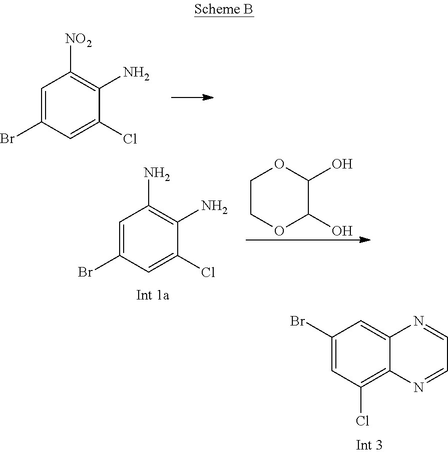

Likewise, 7-bromo-5-chloroquinoxaline (Int 3) is available by applying the same methodology under similar conditions (see Scheme B).

In one particular approach for making compounds of the present invention precursor molecule Int 2 is converted into a compound of formula (III) with Hal2 being bromine and R1 being defined as in the description hereinabove and in the claims by applying C—C coupling reaction conditions.

Typical suitable C—C coupling reactions are, among others, the Heck reaction, the Suzuki coupling, the Stille coupling, the Negishi coupling and coupling reactions utilizing organo cuprates, and well-known variants thereof. Depending on the specific method applied reagents, solvents and reaction conditions are selected accordingly. For instance, in case the introduction of R1 is performed by utilizing Suzuki coupling conditions, precursor molecule Int 2 may be reacted with a suitable borate or boronate ester (B(OSub)₃, with Sub being a suitable substituent, radical or residue) (like trimethylborate or 4,4,5,5-tetramethyl-2-(tetramethyl-1,3,2-dioxaborolan-2-yl)-1,3,2-dioxaborolane) in the presence of an organometallic palladium (II) catalyst (like [1,1′-bis(diphenyl)phosphino)ferrocene]-dichloropalladium(II) dichloromethane complex) and optionally potassium acetate in order to form a derivative of Int 2 in which the bromine substituent is replaced by —B(OH)₂ or —B(OSub)₂, as the case may be; this derivative may then be reacted with a suitable halide R1-Hal in the presence of a palladium(0) complex (e.g., tetrakis(triphenylphosphine)palladium(0)) and a base (e.g., sodium, potassium or cesium carbonate) to build a compound of formula (III).

Similarly, the same compound of formula (III) can be obtained by forming a boron-substituted precursor R1-B(OH)₂ or R1-B(OSub)₂ and reacting it with Int 2 under similar conditions.

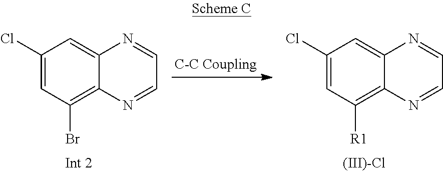

In order to obtain various compounds of formula (I) compounds of formula (III)-Cl obtained as shown in Scheme C may then be subjected to further synthetic modifications for introducing suitable functional groups that allow for, if required, still further modifications.

In a further step compounds of formula (III)-Cl may then be converted into a compound of the invention, i.e. of formula (I) by performing a suitable C—N coupling reaction of the compound of formula (III)-Cl with a compound R2R3HC-NH-RG2, in which RG2 represents a chemical moiety being reactive under the particular C—N coupling reaction. This conversion may be achieved by subjecting the chloride (III)-Cl to a Hartwig-Buchwald reaction, i.e., by reacting it with R2R3HC-NH₂ in the presence of a palladium(II) catalyst, a suitable phosphine ligand and sodium tert.-butylate (e.g., Pd₂(dba)₃/Me₄tBuXPhos/NaOtBu/NH₃). Other C—N coupling reactions may be applied as well. Depending on the specific coupling reaction applied, it may well be that one or both of the reaction partners are subject to chemical transformation into intermediates before the reaction with the appropriate reaction partner occurs; for instance, the suitably substituted halide may be transformed into a respective boronic acid or boronic acid ester derivative before the reaction with the heterocyclic system or the reactive amine derivative occurs. Preferably, this coupling reaction is performed in the presence of a transition metal catalyst. Well-known examples of such C—N coupling reactions are, among others and besides the already mentioned Hartwig-Buchwald reaction, the Ullmann coupling reaction, reactions similar to Suzuki or Heck reaction and coupling reactions utilizing organo cuprates. Depending on the specific method applied reagents, solvents and reaction conditions are selected accordingly.

Another option for preparing compounds of formula (I) may utilize 7-bromo-5-chloroquinoxaline (Int 3) and transform it to a compound of formula (II) by subjecting it to an appropriate C—N-coupling reaction with a compound R2R3HC-NH-RG2, in which RG2 represents a chemical moiety being reactive under the particular C—N coupling reaction. Suitable C—N-coupling reactions are mentioned above and include, without limitation, the Hartwig-Buchwald reaction, the Ullmann coupling reaction, reactions similar to Suzuki or Heck reaction and coupling reactions utilizing organo cuprates. The accordingly obtained compound of formula (II) may then by transformed into a compound of formula (I) by applying a C—C-coupling reaction with a compound R1-RG1, in which RG1 denotes a chemical moiety being reactive under the particular C—C-coupling reactions utilized. Typical suitable C—C coupling reactions are, among others, the Heck reaction, the Suzuki coupling, the Stille coupling, the Negishi coupling and coupling reactions utilizing organo cuprates, and well-known variants thereof. Depending on the specific method applied reagents, solvents and reaction conditions are selected accordingly.

The present invention also refers to a compound of formula (II) or (III) which is a useful intermediate for making compounds of the present invention of formula (I)

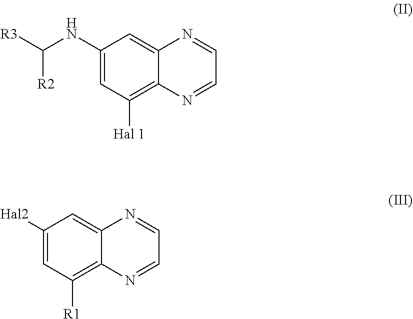

or salts thereof,
wherein
R1, R2 and R3 have the same meaning as defined above or in claims 1 to 7 for compounds of formula (I);
Hal1 and Hal2 both independently from each other denote Cl, Br or

### EXPERIMENTAL PART

### Abbreviations

Some abbreviations that may appear in this application are defined as follows hereinafter:

| Abbreviation | Meaning |
|---|---|
| ¹HNMR | Proton Nuclear Magnetic Resonance |
| aq. | aqueous |
| CDCl₃ | Deuterochloroform |
| d | Doublet |
| dd | Double doublet |
| DMSO-d6 | Hexadeutero-dimethylsulfoxide |
| dt | Double triplet |
| eq. | equivalents |
| h | hour(s) |
| HPLC | High Performance Liquid Chromatography |
| J | Coupling constant |
| LCMS | Liquid Chromatography coupled to Mass Spectrometry |
| m | Multiplet |
| min | Minute |
| mL | Milliliter |
| MTBE | tert-butyl methyl ether |
| n.d. | not determined |
| rH | Relative humidity |
| RT | Room Temperature |
| Rt | Retention time |
| s | Singlet |
| SFC | Supercritical Fluid Chromatography |
| t | Triplet |
| TLC | Thin Layer Chromatography |

The compounds of the present invention can be prepared according to the procedures of the following Schemes and Examples, using appropriate materials and are further exemplified by the following specific examples.

The invention will be illustrated, but not limited, by reference to the specific embodiments described in the following examples. Unless otherwise indicated in the schemes, the variables have the same meaning as described above.

Unless otherwise specified, all starting materials are obtained from commercial suppliers and used without further purifications. Unless otherwise specified, all temperatures are expressed in ° C. and all reactions are conducted at RT. Compounds were purified by either silica chromatography or preparative HPLC.

### LCMS-Analysis:

HPLC/MS-Method:

Gradient: 3.3 min; Flow: 2.4 mL/min von 0 min 4% B, 2.8 min 100% B, 3.3 min 100% B

A: Water+HCOOH (0.05% Vol.); B: Acetonitrile+HCOOH (0.04% Vol.)

Column: Chromolith SpeedROD RP 18e 50-4.6

Wave length: 220 nm

¹HNMR:

Bruker 400 MHz and Bruker 500 MHz

### SYNTHETIC EXAMPLES

### Intermediate 1 (See US2013/116262 A1)

### 3-Bromo-5-chlorobenzene-1,2-diamine

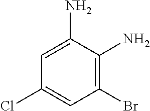

To a stirred solution of tin(II)chloride dihydrate (53.8 g; 238 mmol; 6.00 eq.) in EtOAc (400 mL), 2-bromo-4-chloro-6-nitrophenylamine (10 g; 39.8 mmol; 1.0 eq.) was added in three portions. The reaction was refluxed for 2 h. After this time, the solvent was evaporated and dry residue was suspended in DCM (1 L) and then aqueous solution of NaOH is added (˜300 mL, 10 M, >50 eq.). All reagents were stirred for 4 h and after this time, an organic layer was separated, washed with water and brine and dried over anhydrous Na₂SO₄. Drying agent was filtered off and solvent was evaporated under reduced pressure. 3-Bromo-5-chlorobenzene-1,2-diamine (Intermediate 1) (8.4 g; yield 95%; 97% by UPLC) was obtained as a beige solid and used in the next step without further purification.

### Intermediate 2 (Cf. WO2010/20363 A1)

### 5-bromo-7-chloroquinoxaline

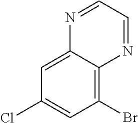 [SMILES: Clc1cc(Br)c2nccnc2c1]

3-bromo-5-chloro-1,2-diaminobenzene Intermediate 1 (8.4 g; 37.9 mmol; 1.0 eq.) was dissolved in EtOH (250 mL) and then 2,3-dihydroxy-1,4-dioxane (4.5 g, 37.9 mmol; 1.0 eq.) was added. The mixture was stirred for 4 h at RT and a second portion of 2,3-dihydroxy-1,4-dioxane (2.3 g; 18.9 mmol; 0.5 eq.) was added. After stirring for 24 h at rt, precipitate was filtered off, washed with EtOH and dried under vacuo to give 5-bromo-7-chloroquinoxaline (Intermediate 2) as a beige solid (6.71 g; yield 74%; 96% by UPLC).

### Intermediate 3 (Cf. WO2010/20363 A1)

### 7-bromo-5-chloroquinoxaline

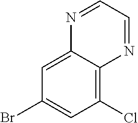 [SMILES: Clc1cc(Br)cc2nccnc12]

5-bromo-3-chloro-1,2-diaminobenzene (4.6 g; 20 mmol; 1.0 eq.) was dissolved in EtOH (200 mL) and then 2,3-dihydroxy-1,4-dioxane (2.5 g, 20 mmol; 1.0 eq.) was added. The mixture was stirred for 4 h at RT and a second portion of 2,3-dihydroxy-1,4-dioxane (1.3 g; 10 mmol; 0.5 eq.) was added. After stirring for 24 h at rt, RM was concentrated in a rotary evaporator and the residue was purified by FCC to provide 7-bromo-5-chloroquinoxaline (Intermediate 3) as a beige solid (4.7 g; yield 92%; 98% by UPLC).

### Intermediate 4

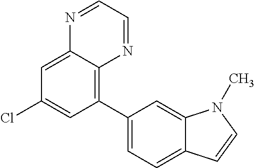 [SMILES: Cn1ccc2ccc(-c3cc(Cl)cc4nccnc34)cc21]

A sealed tube was charged with 5-bromo-7-chloroquinoxaline (Intermediate 2) (3.0 g; 12.2 mmol; 1.0 eq.), 1-methyl-6-(4,4,5,5,-tetramethyl-1,3,2-dioxaborolan-2-yl)-1H-indole (2.5 g; 9.8 mmol; 1.0 eq.), DIPEA (3.2 g; 24.4 mmol; 2.0 eq.), 1,4-dioxane (16 mL) and water (16 mL). The suspension was purged with argon and then Pd(dppf)Cl₂ (0.89 g; 1.22 mmol; 0.10 eq.) was added. RM was sealed and heated at 85° C. for 3 h. After this time, the mixture was filtered through a Celite® pad and the filtrate was diluted with DCM and extracted with water. The organic phase was washed with brine, dried over Na₂SO₄ and then the solvent was evaporated. Crude product was purified by FCC (hexane/EtOAc; gradient) to afford 7-chloro-5-(-1-methyl-1H-indol-6-yl)-quinoxaline (Intermediate 4) (2.2 g; yield 56%; 92% by UPLC) as a yellow solid.

### Intermediate 5

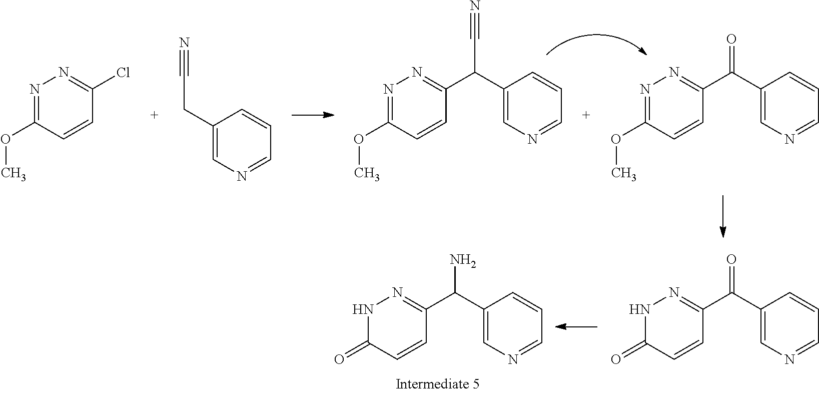

5.1: Pyridin-3-yl-acetonitrile (2.00 g; 16.591 mmol) was dissolved in DMF (20.0 mL) and cooled to 0-5° C. Sodium hydride suspension (60% suspension in paraffin oil) (0.763 g; 19.080 mmol) was added in three portions and the mixture was stirred at 0-5° C. for 30 min. 3-Chloro-6-methoxypyridazine (3.598 g; 24.887 mmol) was added and the mixture stirred at 0° C. for 1 h, then slowly warmed to room temperature and stirred overnight. The reaction mixture was quenched with water (200 mL) and extracted with ethyl acetate. The combined organic layers were washed with brine, dried with sodium sulfate, filtered by suction and evaporated to dryness. The residue (4.85 g; dark red-brown solid), which contained already in parts the product of the following step, was used in the next step without further purification.

5.2: Reaction: Compound 5.1 (4.840 g) was dissolved in acetonitrile (48.4 mL) and the solution cooled to 0-5° C. Potassium tert-butylate (1.188 g; 10.590 mmol) was added in one portion. The mixture was stirred at 0-5° C. for 10 min, then hydrogen peroxide solution (30%; 3.245 ml; 31.770 mmol) was added dropwise. After complete addition the mixture was stirred at 0-5° C. for 15 min, then allowed to warm to room temperature within 15 min and stirred for further 30 min. The reaction mixture was quenched with ice-water (70 mL) and extracted with ethyl acetate. The combined organic layers were washed with brine, dried with sodium sulfate, filtered by suction and evaporated to dryness. The solid residue was triturated with MTBE, filtered by suction and washed with a small amount of MTBE. The filter cake was dried under vacuum at room temperature for 1 h. From the filtrate further compound was isolated by flash chromatography (Companion RF; 80 g Si50 silica gel column). Yield: 1.88 g (82%) pale brown solid; LC/MS, Rt: 1.46 min; (M+H) 216.1

5.3: Compound 5.2 (1.04 g; 4.820 mmol) was suspended in acetic acid (2.0 mL) and hydrogen bromide (32% solution in acetic acid; 4.28 mL; 24.102 mmol) was added dropwise. After 5 min stirring at room temperature a clear brown solution was formed. The mixture was stirred overnight, quenched with ice-water (50 mL) and stirred for 5 min. A clear brown solution was formed, which was alkalified (pH=8-9) with 2N NaOH and then extracted with ethyl acetate. The combined organic layers were washed with brine and a precipitate was formed. It was filtered by suction and washed 2 times with water, once with little acetonitrile and MTBE. The filter cake was dried under high vacuum at 50° C. for 2 h. Yield: 0.60 g (62%) pale brown solid; LC/MS, Rt: 1.13 min; (M+H) 202.1

5.4: To a solution of compound 5.3 (0.60 g; 2.971 mmol) in a solution of ammonia in methanol (7M; 10.610 mL; 74.271 mmol) titanium(IV) isopropoxide (1.823 mL; 5.942 mmol) was added and the resulting mixture was stirred at 55° C. for 66 h. The mixture was cooled to room temperature and filtered by suction. The filter cake was washed with methanol and the filtrate was evaporated to dryness. The solid residue was suspended in dry methanol (5.0 mL), cooled to 0-5° C. and sodium borohydride (0.45 g; 11.883 mmol) was added. The cooling was removed and the mixture was allowed to warm to room temperature within 15 min. The reaction mixture was evaporated to dryness and the residue was purified by flash chromatography (Companion RF; 40 g Si50 silica gel column).

The collected fractions with product were combined and evaporated to dryness. Yield: 124 mg (21%) yellow solid; LC/MS, Rt: 0.38-0.44 min; (M+H) 203.1

### Example 1: 6-{[8-(1-Methyl-1H-indol-6-yl)-quinoxalin-6-ylamino]-pyridin-3-yl-methyl}-2H-pyridazin-3-one

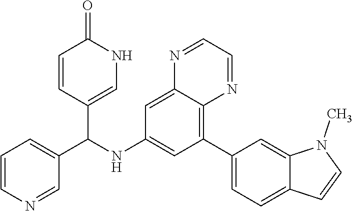 [SMILES: Cn1ccc2ccc(-c3cc(NC(c4cccnc4)c4ccc(=O)[nH]c4)cc4nccnc34)cc21]

Intermediate 4 (125.0 mg; 0.424 mmol), Intermediate 5 (98.7 mg; 0.488 mmol), Tris-(dibenzylidenacetone)-dipalladium (38.9 mg; 0.042 mmol), racemic-2,2′-Bis(diphenylphosphino)-1,1′-binaphthalene (52.8 mg; 0.085 mmol) and sodium tert-butylate (122.3 mg; 1.273 mmol) were suspended in dry toluene (8.0 mL). The vial was sealed with a septum and argon was bubbled through the reaction mixture for 5 min. The mixture was heated to 100° C. and stirred overnight. The reaction mixture was diluted with water (30 mL) and extracted with ethyl acetate. The combined organic layers were washed with brine, dried with sodium sulfate, filtered by suction and evaporated to dryness. The oily residue was purified in by chromatography (Companion RF; 40 g Si50 silica gel column). The solid residue was suspended in ethyl acetate-methanol (95:5), filtered by suction, washed with little MTBE and dried under vacuum at 50° C. for 3 h. Yield: 39 mg (20%) yellow solid; LC/MSW, Rt: 1.77 min; (M+H) 460.1; ¹H NMR (500 MHz, DMSO-d₆) δ 13.01 (d, J=2.3 Hz, 1H), 8.77 (d, J=2.2 Hz, 1H), 8.64 (d, J=1.9 Hz, 1H), 8.52 (dd, J=4.8, 1.6 Hz, 1H), 8.50 (d, J=1.9 Hz, 1H), 7.93-7.90 (m, 1H), 7.63-7.58 (m, 3H), 7.58 (d, J=2.6 Hz, 1H), 7.56 (d, J=7.6 Hz, 1H), 7.45-7.41 (m, 1H), 7.38 (d, J=3.1 Hz, 1H), 7.27 (dd, J=8.1, 1.5 Hz, 1H), 6.94 (dd, J=9.8, 2.2 Hz, 1H), 6.90 (d, J=2.6 Hz, 1H), 6.47 (dd, J=3.1, 0.9 Hz, 1H), 6.03 (d, J=7.6 Hz, 1H), 3.81 (s, 3H).

### Chiral Separation of 6-{[8-(1-Methyl-1H-indol-6-yl)-quinoxalin-6-ylamino]-pyridin-3-yl-methyl}-2H-pyridazin-3-one (Example 1) into the Enantiomers (Conformation was Arbitrarily Assigned)

### Example 2: 6-{(S)-[8-(1-Methyl-1H-indol-6-yl)-quinoxalin-6-ylamino]-pyridin-3-yl-methyl}-2H-pyridazin-3-one

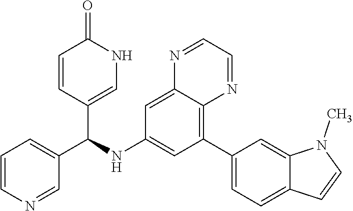 [SMILES: Cn1ccc2ccc(-c3cc(N[C@@H](c4cccnc4)c4ccc(=O)[nH]c4)cc4nccnc34)cc21]

### Example 3: 6-{(R)-[8-(1-Methyl-1H-indol-6-yl)-quinoxalin-6-ylamino]-pyridin-3-yl-methyl}-2H-pyridazin-3-one

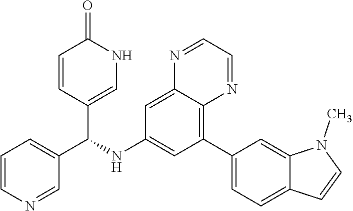 [SMILES: Cn1ccc2ccc(-c3cc(N[C@H](c4cccnc4)c4ccc(=O)[nH]c4)cc4nccnc34)cc21]

The preparative separation of example 1 (39 mg) was performed by SFC (column: ChiralPak AD-H; eluent: CO₂:2-propanol (containing 0.5% diethyl-amine) −55:45). The combined fractions were evaporated to dryness. The oily residues were dissolved in acetonitrile, diluted with water and lyophilized.

Example 2: 19 mg yellow solid; LC/MS, Rt: 1.77 min; (M+H) 460.1; ¹H NMR (500 MHz, DMSO-d₆) δ 13.01 (d, J=2.3 Hz, 1H), 8.77 (d, J=2.2 Hz, 1H), 8.64 (d, J=1.9 Hz, 1H), 8.52 (dd, J=4.8, 1.6 Hz, 1H), 8.50 (d, J=1.9 Hz, 1H), 7.93-7.90 (m, 1H), 7.63-7.58 (m, 3H), 7.58 (d, J=2.6 Hz, 1H), 7.56 (d, J=7.6 Hz, 1H), 7.45-7.41 (m, 1H), 7.38 (d, J=3.1 Hz, 1H), 7.27 (dd, J=8.1, 1.5 Hz, 1H), 6.94 (dd, J=9.8, 2.2 Hz, 1H), 6.90 (d, J=2.6 Hz, 1H), 6.47 (dd, J=3.1, 0.9 Hz, 1H), 6.03 (d, J=7.6 Hz, 1H), 3.81 (s, 3H).

Example 3: 19 mg yellow solid; LC/MS, Rt: 1.77 min; (M+H) 460.1; ¹H NMR (500 MHz, DMSO-d₆) δ 13.01 (d, J=2.3 Hz, 1H), 8.77 (d, J=2.2 Hz, 1H), 8.64 (d, J=1.9 Hz, 1H), 8.52 (dd, J=4.8, 1.6 Hz, 1H), 8.50 (d, J=1.9 Hz, 1H), 7.93-7.90 (m, 1H), 7.63-7.58 (m, 3H), 7.58 (d, J=2.6 Hz, 1H), 7.56 (d, J=7.6 Hz, 1H), 7.45-7.41 (m, 1H), 7.38 (d, J=3.1 Hz, 1H), 7.27 (dd, J=8.1, 1.5 Hz, 1H), 6.94 (dd, J=9.8, 2.2 Hz, 1H), 6.90 (d, J=2.6 Hz, 1H), 6.47 (dd, J=3.1, 0.9 Hz, 1H), 6.03 (d, J=7.6 Hz, 1H), 3.81 (s, 3H).

### Intermediate 6

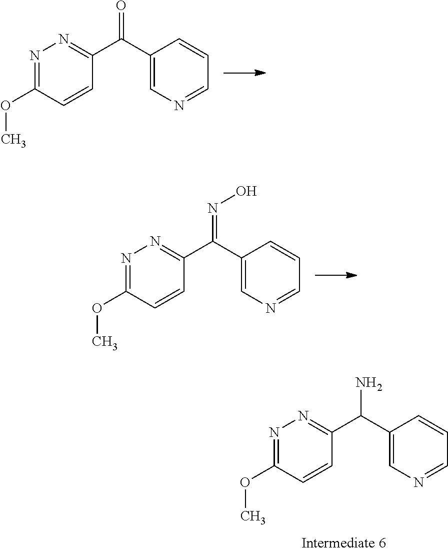 [SMILES: COc1ccc(C(=O)c2cccnc2)nn1 ; COc1ccc(/C(=N/O)c2cccnc2)nn1 ; COc1ccc(C(N)c2cccnc2)nn1]

6.1: (6-Methoxy-pyridazin-3-yl)-pyridin-3-yl-methanone (4.23 g; 19.655 mmol), hydroxylammonium chloride (1.90 g; 27.342 mmol) and sodium acetate trihydrate (3.70 g; 27.190 mmol) were dissolved in a mixture of ethanol (70.0 mL) and water (35.0 mL) and stirred at 88° C. overnight. A light brown solution with yellow precipitate was formed. The reaction mixture was evaporated to an aqueous residue and the precipitate was filtered off by suction, washed twice with little water and once with little acetonitrile and dried under vacuum at 50° C. for 5 h. Yield: 3.93 g (83%) yellow solid; LC/MS, Rt: 0.39-1.07 min; (M+H) 231.2

6.2: Compound 6.1 (3.93 g; 17.070 mmol) and ammonium acetate (1.97 g; 25.606 mmol) were suspended in ethanol (40.0 mL) and ammonia hydroxide (32%; 40.0 mL). Zinc dust (2.46 g; 37.555 mmol) was added at room temperature and the mixture was stirred at 20-30° C. for 1 h. A brown solution with a pale brown precipitate was formed. Further zinc dust (0.893 g; 13.656 mmol) was added and the mixture was stirred for another 1 h at 20-30° C. and 1 h at room temperature. The reaction mixture was filtered by suction and the residue was washed twice with ethanol/water (1:1). The filtrate was evaporated to an aqueous residue. The aqueous residue was extracted 4 times with ethyl acetate (100 mL) and once with tert-butanol (100 mL). The combined organic layers were dried with sodium sulfate, filtered by suction and evaporated to dryness. The residue was purified by flash chromatography (Companion RF; 80 g Si50 silica gel column). Yield: 1.04 g dark brown oil; LC/MS, Rt: 0.39-0.71 min; (M+H) 217.2

### Example 4: [(6-Methoxy-pyridazin-3-yl)-pyridin-3-yl-methyl]-[8-(1-methyl-1H-indol-6-yl)-quinoxalin-6-yl]-amine

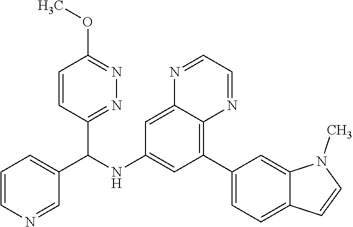 [SMILES: COc1ccc(C(Nc2cc(-c3ccc4ccn(C)c4c3)c3nccnc3c2)c2cccnc2)nn1]

Preparation as described for example 1 using Intermediate 4 (150.0 mg; 0.511 mmol), Intermediate 6 (179.1 mg; 0.766 mmol), Tris-(dibenzyliden-acetone)-dipalladium (46.8 mg; 0.051 mmol), racemic-2,2′-Bis(diphenylphos-phino)-1,1′-binaphthalene (63.6 mg; 0.102 mmol) and sodium tert-butylate (147.2 mg; 1.532 mmol). Reaction time: 2 h; Purification: Flash chromatography (Companion RF; 24 g Si50 silica gel column) and chromatography (Companion RF; 35 g C18HC column). The combined fractions were evaporated to an aqueous residue, alkalified with saturated NaHCO₃ solution and extracted 3 times with ethyl acetate. The combined organic layers were washed with brine, dried with sodium sulfate, filtered by suction and evaporated to dryness. Yield: 159 mg (66%) oil; LC/MS, Rt: 2.01 min; (M+H) 474.2

### Chiral Separation of [(6-Methoxy-pyridazin-3-yl)-pyridin-3-yl-methyl]-[8-(1-methyl-1H-indol-6-yl)-quinoxalin-6-yl]-amine (Example 4) into the Enantiomers (Conformation was Arbitrarily Assigned)

### Example 5: [(S)-(6-Methoxy-pyridazin-3-yl)-pyridin-3-yl-methyl]-[8-(1-methyl-1H-indol-6-yl)-quinoxalin-6-yl]-amine

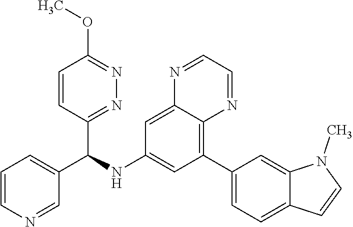 [SMILES: COc1ccc([C@@H](Nc2cc(-c3ccc4ccn(C)c4c3)c3nccnc3c2)c2cccnc2)nn1]

### Example 6: [(R)-(6-Methoxy-pyridazin-3-yl)-pyridin-3-yl-methyl]-[8-(1-methyl-1H-indol-6-yl)-quinoxalin-6-yl]-amine

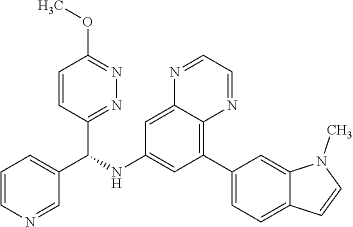 [SMILES: COc1ccc([C@H](Nc2cc(-c3ccc4ccn(C)c4c3)c3nccnc3c2)c2cccnc2)nn1]

The preparative separation of Example 4 (159 mg) was performed by preparative HPLC (column: ChiralPak IA; eluent: ethanol. The combined fractions were evaporated to dryness. The oily residues were dissolved in acetonitrile, diluted with water and lyophilized.

Example 5: 59 mg yellow solid; LC/MS, Rt: 2.01 min; (M+H) 474.2; ¹H NMR (500 MHz, DMSO-d₆) δ 8.82 (d, J=2.2 Hz, 1H), 8.62 (d, J=1.9 Hz, 1H), 8.51 (dd, J=4.8, 1.6 Hz, 1H), 8.49 (d, J=1.9 Hz, 1H), 7.97-7.93 (m, 1H), 7.84 (d, J=9.1 Hz, 1H), 7.72 (d, J=7.4 Hz, 1H), 7.66 (d, J=2.7 Hz, 1H), 7.63-7.61 (m, 1H), 7.60 (d, J=8.1 Hz, 1H), 7.43-7.40 (m, 1H), 7.38 (d, J=3.1 Hz, 1H), 7.29-7.25 (m, 2H), 6.88 (d, J=2.6 Hz, 1H), 6.48-6.46 (m, 1H), 6.30 (d, J=7.3 Hz, 1H), 4.03 (s, 3H), 3.81 (s, 3H).

Example 6: 54 mg yellow solid; LC/MS, Rt: 2.02 min; (M+H) 474.2; ¹H NMR (500 MHz, DMSO-d₆) δ 8.82 (d, J=2.1 Hz, 1H), 8.62 (d, J=1.9 Hz, 1H), 8.51 (dd, J=4.8, 1.6 Hz, 1H), 8.49 (d, J=1.9 Hz, 1H), 7.97-7.93 (m, 1H), 7.84 (d, J=9.2 Hz, 1H), 7.72 (d, J=7.3 Hz, 1H), 7.66 (d, J=2.6 Hz, 1H), 7.63-7.61 (m, 1H), 7.60 (d, J=8.1 Hz, 1H), 7.43-7.40 (m, 1H), 7.38 (d, J=3.1 Hz, 1H), 7.30-7.25 (m, 2H), 6.88 (d, J=2.5 Hz, 1H), 6.48-6.46 (m, 1H), 6.30 (d, J=7.3 Hz, 1H), 4.03 (s, 3H), 3.81 (s, 3H).

### Intermediate 7

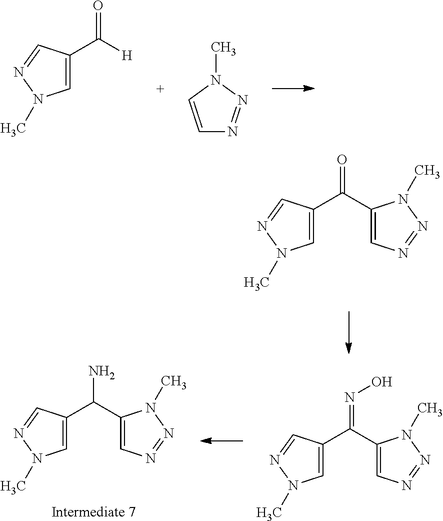

7.1: 1-Methyl-1H-[1,2,3]triazole (1.50 g; 17.150 mmol) was dissolved in dry THF (30.0 mL) under argon and cooled to −65° C. Butyllithium (2.5 M in hexane; 6.86 mL; 17.150 mmol) was added dropwise over a period of 10 min. The temperature was hold between −65° C. and −60° C. A colorless suspension was formed, which was stirred at −65° C. for 1 h. 1-Methyl-1H-pyrazole-4-carbaldehyde (1.89 g; 17.150 mmol), dissolved in dry THF (10.0 mL), was added dropwise at −65° C. and the mixture stirred at −65° C. for 15 min. The reaction mixture was slowly warmed to 0° C. within 1.5 h and then quenched with methanol (10 mL) and evaporated to dryness. The crude residue was dissolved in dry THF (50.0 mL) and water (15.0 mL). Manganese(IV) oxide (2.98 g; 34.299 mmol) was added, and the mixture was stirred at 80° C. overnight. Manganese(IV) oxide (2.98 g; 34.299 mmol) was added and the mixture was stirred for further 24 h at 80° C. The mixture was filtered over kieselguhr. The residue was washed with dichloro-methane/methanol (30%). The combined filtrates were evaporated to an aqueous residue. A precipitate was formed, which was filtered by suction and washed with little water, acetonitrile and MTBE and dried under vacuum at 50° C. for 1 h. The filtrate was diluted with water and extracted with ethyl acetate. The combined organic layers were washed with sodium sulfate, filtered by suction and evaporated to dryness. The solid residue was triturated with little acetonitrile, filtered by suction and washed with little acetonitrile and MTBE, and dried under vacuum at 50° C. for 1 h. Yield: 2.38 g colorless solid; LC/MS, Rt: 1.29 min; (M+H) 192.2

7.2: Compound 7.1 (2.38 g; 12.448 mmol), hydroxylammonium chloride (1.73 g; 24.897 mmol) and sodium acetate trihydrate (3.39 g; 24.897 mmol) were suspended in ethanol (70.0 mL) and water (30.0 mL) and refluxed overnight. The reaction mixture was evaporated to half of the volume and ethyl acetate (100 mL) was added. The organic layer was separated and the aqueous layer was extracted 2 times with ethyl acetate (50 mL). The combined organic layers were washed with brine, dried with sodium sulfate, filtered by suction and evaporated to dryness. The solid residue was triturated with ethyl acetat-MTBE (1:1), filtered by suction, washed with MTBE, and dried under vacuum at 50° C. for 3 h. Yield: 2.83 g pale-green solid; LC/MS, Rt: 0.47 min (M+H) 207.1

7.3: Reduction of compound 7.2 was performed as described for compound 6.2. Yield: 1.85 g (68%) yellow oil; LC/MS, Rt: 0.38-0.45 min; (M+H) 193.1

### Example 7: [8-(1-Methyl-1H-indol-6-yl)-quinoxalin-6-yl]-[(1-methyl-1H-pyraz-ol-4-yl)-(3-methyl-3H-[1,2,3]triazol-4-yl)-methyl]-amine

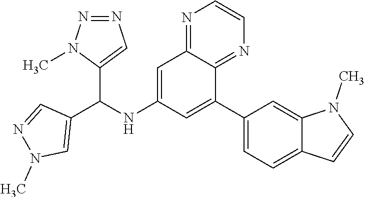 [SMILES: Cn1cc(C(Nc2cc(-c3ccc4ccn(C)c4c3)c3nccnc3c2)c2cnnn2C)cn1]

Preparation as described for Example 1 using intermediate 4 (125.0 mg; 0.424 mmol), intermediate 7 (122.3 mg; 0.636 mmol), Tris-(dibenzyliden-acetone)-dipalladium (38.9 mg; 0.042 mmol), racemic-2,2′-Bis(diphenylphosphino)-1,1′-binaphthalene (52.8 mg; 0.085 mmol) and sodium tert-butylate (122.3 mg; 1.273 mmol). Purification by preparative HPLC (Agilent 1260 HPLC; column: Waters SunFire C18 5 μM 30×150 mm) and by flash chromatography (Companion RF; 24 g Si50 silica gel column). Yield: 97 mg (51%) yellow foam; LC/MS, Rt: 1.98 min; (M+H) 450.2

### Chiral Separation of [8-(1-Methyl-1H-indol-6-yl)-quinoxalin-6-yl]-[(1-methyl-1H-pyrazol-4-yl)-(3-methyl-3H-[1,2,3]triazol-4-yl)-methyl]-amine (Example 7) into the Enantiomers (Conformation was Arbitrarily Assigned)

### Example 8: [8-(1-Methyl-1H-indol-6-yl)-quinoxalin-6-yl]-[(S)-(1-methyl-1H-pyrazol-4-yl)-(3-methyl-3H-[1,2,3]triazol-4-yl)-methyl]-amine

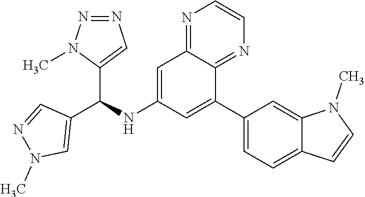 [SMILES: Cn1cc([C@H](Nc2cc(-c3ccc4ccn(C)c4c3)c3nccnc3c2)c2cnnn2C)cn1]

### Example 9: [8-(1-Methyl-1H-indol-6-yl)-quinoxalin-6-yl]-[(R)-(1-methyl-1H-pyrazol-4-yl)-(3-methyl-3H-[1,2,3]triazol-4-yl)-methyl]-amine

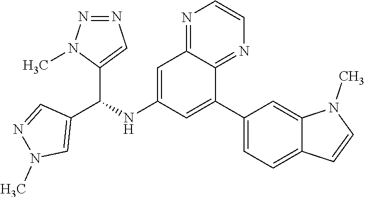 [SMILES: Cn1cc([C@@H](Nc2cc(-c3ccc4ccn(C)c4c3)c3nccnc3c2)c2cnnn2C)cn1]

The preparative separation of Example 7 (97 mg) was performed by SFC (column: ChiralPak AD-H; eluent: CO₂:methanol −60:40). The combined fractions were evaporated to dryness. The oily residues were dissolved in acetonitrile, diluted with water and lyophilized.

Example 8: 41 mg yellow solid; LC/MS, Rt: 1.97-1.98 min; (M+H) 450.2; ¹H NMR (500 MHz, DMSO-d₆) δ 8.64 (d, J=1.9 Hz, 1H), 8.50 (d, J=1.9 Hz, 1H), 7.71 (s, 1H), 7.62-7.58 (m, 2H), 7.55 (s, 1H), 7.51 (d, J=2.7 Hz, 1H), 7.48 (d, J=0.8 Hz, 1H), 7.38 (d, J=3.0 Hz, 1H), 7.30 (d, J=7.6 Hz, 1H), 7.26 (dd, J=8.1, 1.6 Hz, 1H), 6.95 (d, J=2.6 Hz, 1H), 6.47 (dd, J=3.1, 0.8 Hz, 1H), 6.24 (d, J=7.5 Hz, 1H), 4.00 (s, 3H), 3.82 (s, 3H), 3.81 (s, 3H).

Example 9: 41 mg yellow solid; LC/MS, Rt: 1.97-1.98 min; (M+H) 450.2; ¹H NMR (500 MHz, DMSO-d₆) δ 8.64 (d, J=1.9 Hz, 1H), 8.50 (d, J=1.9 Hz, 1H), 7.71 (s, 1H), 7.62-7.58 (m, 2H), 7.55 (s, 1H), 7.51 (d, J=2.6 Hz, 1H), 7.48 (d, J=0.8 Hz, 1H), 7.38 (d, J=3.1 Hz, 1H), 7.30 (d, J=7.6 Hz, 1H), 7.26 (dd, J=8.1, 1.6 Hz, 1H), 6.95 (d, J=2.5 Hz, 1H), 6.47 (dd, J=3.1, 0.8 Hz, 1H), 6.24 (d, J=7.5 Hz, 1H), 4.00 (s, 3H), 3.82 (s, 3H), 3.81 (s, 3H).

### Intermediate 8

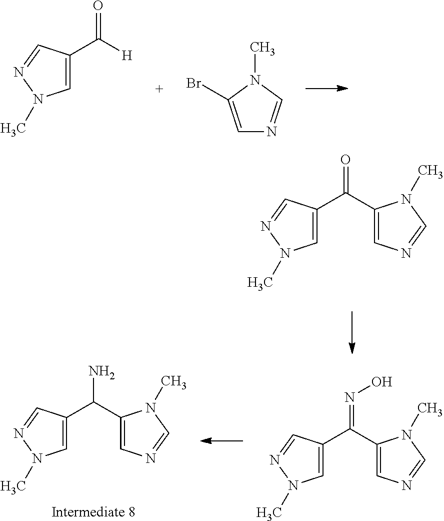

8.1: 5-Bromo-1-methyl-1H-imidazole (2.83 g; 17.028 mmol) was dissolved in dy THF (25.0 mL) under argon and cooled to 0° C. Lithium chloro(isopropyl)-magnesium chloride (10.48 mL; 13.622 mmol) was added dropwise within 15 min. A colorless suspension was formed, which was stirred at 0° C. for 1 h. The suspension was added at 0° C. via a syringe to a solution of 1-methyl-1H-pyrazole-4-carbaldehyde (1.50 g; 13.622 mmol) in dry THF (10.0 mL) and the mixture was stirred at 0° C. for 30 min. The mixture slowly warmed to room temperature and stirred overnight. The reaction mixture was cooled to 0-5° C., quenched with methanol (10 mL) and evaporated to dryness. The oily residue was dissolved in methanol (30.0 mL), Manganese(IV) oxide (4.74 g; 54.489 mmol) was added and the mixture was refluxed overnight. The reaction mixture was filtered through kieselguhr and the residue washed 3 times with methanol. The filtrate was evaporated to dryness and the residue purified by flash chromatography (Companion RF; 120 g Si50 silica gel column). Yield: 517 mg (19%) yellow oil; LC/MS, 0.402 min; (M+H) 191.2 Steps 8.2 and 8.3: Formation of the oxime and subsequent reduction to the amine was performed as described in 7.2 and 7.3 respectively. Yield: 0.44 g (100%) yellow oil; LC/MS, Rt: 0.37 min; (M+H) 192.2

### Example 10: [(3-Methyl-3H-imidazol-4-yl)-(1-methyl-1H-pyrazol-4-yl)-methyl]-[8-(1-methyl-1H-indol-6-yl)-quinoxalin-6-yl]-amine

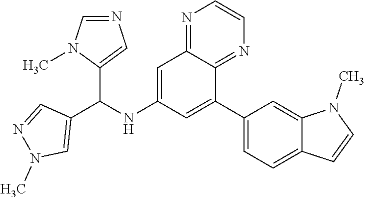 [SMILES: Cn1cc(C(Nc2cc(-c3ccc4ccn(C)c4c3)c3nccnc3c2)c2cncn2C)cn1]

Preparation as described for Example 1 using Intermediate 4 (125.0 mg; 0.426 mmol), Intermediate 8 (98.1 mg; 0.511 mmol), Tris-(dibenzyliden-acetone)-dipalladium (39.0 mg; 0.043 mmol), racemic-2,2′-Bis(diphenyl-phosphino)-1,1′-binaphthalene (53.0 mg; 0.085 mmol) and sodium tert-butylate (122.7 mg; 1.277 mmol). Purification by flash chromatography (Companion RF 40 g Si50 silica gel column). Yield: 76 mg (40%) yellow foam; LC/MS, Rt: 1.64 min; (M+H) 449.2

### Chiral Separation of [(3-Methyl-3H-imidazol-4-yl)-(1-methyl-1H-pyrazol-4-yl)-methyl]-[8-(1-methyl-1H-indol-6-yl)-quinoxalin-6-yl]-amine (Example 10) into the Enantiomers (Please Note: Conformation was Arbitrarily Assigned)

### Example 11: [(S)-(3-Methyl-3H-imidazol-4-yl)-(1-methyl-1H-pyrazol-4-yl)-methyl]-[8-(1-methyl-1H-indol-6-yl)-quinoxalin-6-yl]-amine

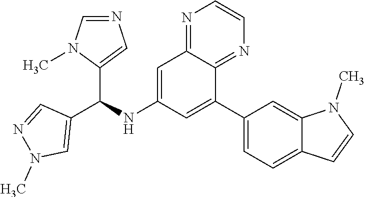 [SMILES: Cn1cc([C@H](Nc2cc(-c3ccc4ccn(C)c4c3)c3nccnc3c2)c2cncn2C)cn1]

### Example 12: [(R)-(3-Methyl-3H-imidazol-4-yl)-(1-methyl-1H-pyrazol-4-yl)-methyl]-[8-(1-methyl-1H-indol-6-yl)-quinoxalin-6-yl]-amine

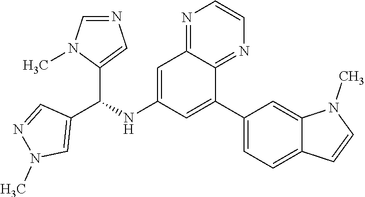 [SMILES: Cn1cc([C@@H](Nc2cc(-c3ccc4ccn(C)c4c3)c3nccnc3c2)c2cncn2C)cn1]

The preparative separation of example 10 (125 mg) was performed by SFC (column: ChiralPak AD-H; eluent: CO₂:methanol (containing 0.5% diethyl-amine) −65:35). The combined fractions were evaporated to dryness. The oily residues were dissolved in acetonitrile, diluted with water and lyophilized.

Example 11: 53 mg yellow solid; LC/MS, Rt: 1.19 min; (M+H) 449.2; ¹H NMR (500 MHz, DMSO-d₆) δ 8.62 (d, J=1.9 Hz, 1H), 8.46 (d, J=1.9 Hz, 1H), 7.67 (s, 1H), 7.61-7.58 (m, 3H), 7.51 (d, J=2.6 Hz, 1H), 7.45 (s, 1H), 7.37 (d, J=3.1 Hz, 1H), 7.25 (dd, J=8.2, 1.4 Hz, 1H), 7.18 (d, J=7.4 Hz, 1H), 6.94 (d, J=2.6 Hz, 1H), 6.67-6.65 (m, 1H), 6.48-6.46 (m, 1H), 5.99 (d, J=7.3 Hz, 1H), 3.82 (s, 3H), 3.81 (s, 3H), 3.61 (s, 3H).

Example 12: 52 mg yellow solid; LC/MS, Rt: 1.16 min; (M+H) 449.2; ¹H NMR (500 MHz, DMSO-d₆) δ 8.62 (d, J=1.9 Hz, 1H), 8.46 (d, J=1.9 Hz, 1H), 7.67 (s, 1H), 7.61-7.58 (m, 3H), 7.51 (d, J=2.6 Hz, 1H), 7.46-7.44 (m, 1H), 7.37 (d, J=3.1 Hz, 1H), 7.25 (dd, J=8.2, 1.4 Hz, 1H), 7.18 (d, J=7.4 Hz, 1H), 6.94 (d, J=2.5 Hz, 1H), 6.67-6.65 (m, 1H), 6.47 (dd, J=3.0, 0.8 Hz, 1H), 5.99 (d, J=7.3 Hz, 1H), 3.82 (s, 3H), 3.81 (s, 3H), 3.61 (s, 3H).

### Intermediate 9

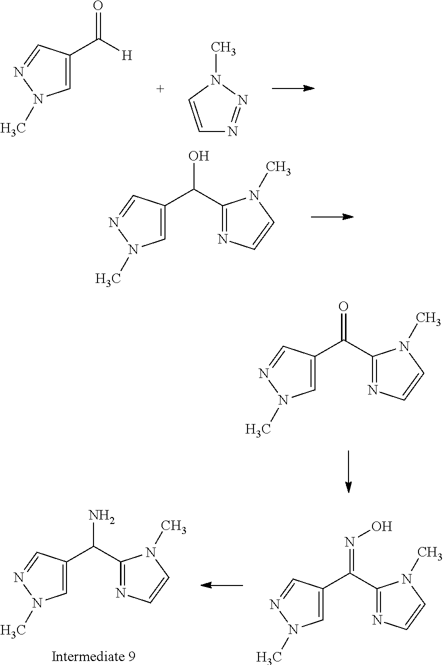

9.1: 1-Methylimidazole (2.00 g; 23.141 mmol) was dissolved in dry diethyl ether dried (25.0 mL) under argon and the solution was cooled to −65° C. Butyllithium (2.5 M in hexane; 9.257 mL; 23.141 mmol) was added dropwise within 15 min. The temperature was kept between −65° C. and −60° C. A colorless suspension was formed, which was stirred at −65° C. for 1 h. 1-Methyl-1H-pyrazole-4-carbaldehyde (2.55 g; 23.141 mmol), dissolved in dry diethyl ether (10.0 mL) was added slowly. The temperature was kept 15 min at −65° C. and then the reaction mixture was slowly warmed to room temperature and stirred overnight. The reaction mixture was cooled to 0° C. and quenched with saturated NH₄Cl solution (4 mL). The mixture was diluted with water (10 mL) and extracted with ethyl acetate. The aqueous layer was evaporated to dryness and the residue triturated 2 times with dichloro-methane/10% methanol (100 mL). This solution was combined with the ethyl acetate extract, dried with sodium sulfate, filtered by suction and evaporated to dryness. Yield: 4.46 g brown oil (100%); LC/MS, Rt: 0.40 min; (M+H) 193.2 9.2: Compound 9.1 (4.46 g; 23.203 mmol) was dissolved in THF (25.0 mL) and water (5.0 mL). MnO₂ (14.26 g; 46.406 mmol) was added and the mixture was stirred at 60° C. for 1 h. The hot mixture was filtered through kieselguhr and the residue washed with THF/methanol (1:1). The filtrate was evaporated to dryness. Yield: 3.19 g (72%) red solid/oil; LC/MS, Rt: 1.3 min; (M+H) 191.2

Steps 9.3 and 9.4: Formation of the oxime and subsequent reduction to the amine was performed as described in 7.2 and 7.3 respectively. Yield: 1.14 g red oil; LC/MS, Rt: 0.37-0.46 min; (M+H−NH₂) 175.2

### Example 13: [(1-Methyl-1H-imidazol-2-yl)-(1-methyl-1H-pyrazol-4-yl)-methyl]-[8-(1-methyl-1H-indol-6-yl)-quinoxalin-6-yl]-amine

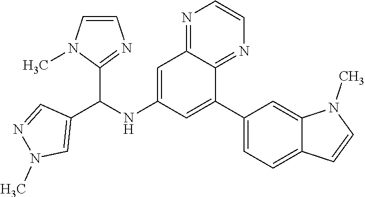 [SMILES: Cn1cc(C(Nc2cc(-c3ccc4ccn(C)c4c3)c3nccnc3c2)c2nccn2C)cn1]

Preparation as described for Example 1 using Intermediate 4 (125.0 mg; 0.424 mmol), Intermediate 9 (130.0 mg; 0.636 mmol), Tris-(dibenzyliden-acetone)-dipalladium (38.9 mg; 0.042 mmol), racemic-2,2′-Bis(diphenylphos-phino)-1,1′-binaphthalene (52.8 mg; 0.085 mmol) and sodium tert-butylate (122.3 mg; 1.273 mmol). Purification by flash chromatography (Companion RF; 100 g C18 silica gel column and Companion RF; 40 g Si50 silica gel column). Yield: 119 mg (63%) yellow foam; LC/MS, Rt: 1.64 min; (M+H) 449.2

### Chiral Separation of [(1-Methyl-1H-imidazol-2-yl)-(1-methyl-1H-pyrazol-4-yl)-methyl]-[8-(1-methyl-1H-indol-6-yl)-quinoxalin-6-yl]-amine (Example 13) into the Enatiomers (Conformation was Arbitrarily Assigned)

### Example 14: [(S)-(1-Methyl-1H-imidazol-2-yl)-(1-methyl-1H-pyrazol-4-yl)-methyl]-[8-(1-methyl-1H-indol-6-yl)-quinoxalin-6-yl]-amine

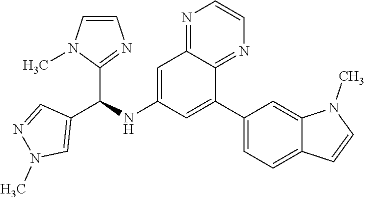 [SMILES: Cn1cc([C@H](Nc2cc(-c3ccc4ccn(C)c4c3)c3nccnc3c2)c2nccn2C)cn1]

### Example 15: [(R)-(1-Methyl-1H-imidazol-2-yl)-(1-methyl-1H-pyrazol-4-yl)-methyl]-[8-(1-methyl-1H-indol-6-yl)-quinoxalin-6-yl]-amine

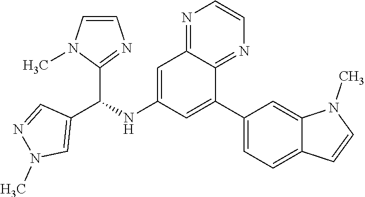 [SMILES: Cn1cc([C@@H](Nc2cc(-c3ccc4ccn(C)c4c3)c3nccnc3c2)c2nccn2C)cn1]

The preparative separation of Example 13 (117 mg) was performed by SFC (column: ChiralPak AD-H; eluent: CO₂:2-propanol (containing 0.5% diethyl-amine) −60:40). The combined fractions were evaporated to dryness. The oily residues were dissolved in acetonitrile, diluted with water and lyophilized.

Example 14: 49.5 mg yellow solid; LC/MS, Rt: 1.64 min; (M+H) 449.2; ¹H NMR (400 MHz, DMSO-d₆) δ 8.62 (d, J=2.0 Hz, 1H), 8.46 (d, J=1.9 Hz, 1H), 7.67 (s, 1H), 7.61-7.57 (m, 3H), 7.45-7.43 (m, 1H), 7.37 (d, J=3.1 Hz, 1H), 7.26 (dd, J=8.2, 1.4 Hz, 1H), 7.22 (d, J=7.3 Hz, 1H), 7.08 (d, J=1.2 Hz, 1H), 6.96 (d, J=2.6 Hz, 1H), 6.83 (d, J=1.2 Hz, 1H), 6.47 (dd, J=3.0, 0.9 Hz, 1H), 6.04 (d, J=7.2 Hz, 1H), 3.81 (s, 3H), 3.80 (s, 3H), 3.66 (s, 3H).

Example 15: 51 mg yellow solid; LC/MS, Rt: 1.64 min; (M+H) 449.2; ¹H NMR (500 MHz, DMSO-d₆) δ 8.62 (d, J=1.9 Hz, 1H), 8.46 (d, J=1.9 Hz, 1H), 7.67 (s, 1H), 7.61-7.58 (m, 3H), 7.45-7.43 (m, 1H), 7.37 (d, J=3.1 Hz, 1H), 7.26 (dd, J=8.2, 1.4 Hz, 1H), 7.22 (d, J=7.2 Hz, 1H), 7.08 (d, J=1.2 Hz, 1H), 6.96 (d, J=2.5 Hz, 1H), 6.84 (d, J=1.1 Hz, 1H), 6.48-6.46 (m, 1H), 6.04 (d, J=7.1 Hz, 1H), 3.81 (s, 3H), 3.80 (s, 3H), 3.66 (s, 3H).

### Intermediate 10

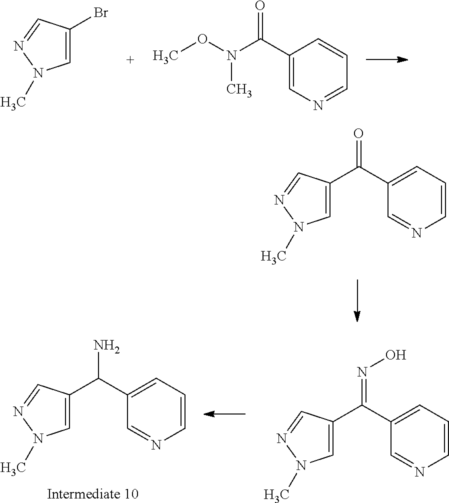

10.1: 4-Bromo-1-methyl-1H-pyrazole (1.40 g; 8.405 mmol) was dissolved in dry THF (15.0 mL) under argon and cooled to −65° C. Butyllithium (2.5 M solution in n-hexane; 3.70 mL; 9.245 mmol) was added dropwise within 10 min. A colorless precipitate was formed. The suspension was stirred at −65° C. for 30 min and then added dropwise under argon to a solution of N-methoxy-N-methyl-nicotinamide (1.50 g; 7.636 mmol) in dry THF (5.0 mL) at −65° C. within 10 min. A yellow suspension was formed, which was stirred at −65° C. for 20 min and then warmed to −10° C. within 45 min. The reaction mixture was quenched with 10% citric acid solution (3 mL), diluted with MTBE (50 mL), washed with water and brine, dried with sodium sulfate, filtered and evaporated to dryness. The solid residue was triturated with MTBE, filtered by suction, washed with little MTBE and dried. Yield: 942 mg (66%) colorless solid; LC/MS, Rt: 1.19 min; (M+H) 188.2

Steps 10.2 and 10.3: Formation of the oxime and subsequent reduction to the amine was performed as described in 7.2 and 7.3 respectively. Yield: 3.00 g (83%) brown solid; LC/MS, Rt: 0.38-0.47 min; (M+H) 189.2

### Example 16: [8-(1-Methyl-1H-indol-6-yl)-quinoxalin-6-yl]-[(1-methyl-1H-pyrazol-4-yl)-pyridin-3-yl-methyl]-amine

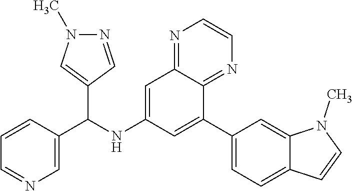 [SMILES: Cn1cc(C(Nc2cc(-c3ccc4ccn(C)c4c3)c3nccnc3c2)c2cccnc2)cn1]

Preparation as described for Example 1 using Intermediate 4 (100.0 mg; 0.335 mmol), Intermediate 10 (99.2 mg; 0.502 mmol), Tris-(dibenzyliden-acetone)-dipalladium (31.3 mg; 0.033 mmol), racemic-2,2′-Bis(diphenyl-phosphino)-1,1′-binaphthalene (42.6 mg; 0.067 mmol) and sodium tert-butylate (97.6 mg; 1.005 mmol). Purification by flash chromatography (CombiFlashRF 200). Yield: 140 mg (94%) yellow foam; LC/MS, Rt: 1.83 min; (M+H) 446.2

### Chiral Separation of [8-(1-Methyl-1H-indol-6-yl)-quinoxalin-6-yl]-[(1-methyl-1H-pyrazol-4-yl)-pyridin-3-yl-methyl]-amine (Example 16) (Conformation was Arbitrarily Assigned)

### Example 17: [8-(1-Methyl-1H-indol-6-yl)-quinoxalin-6-yl]-[(S)-(1-methyl-1H-pyrazol-4-yl)-pyridin-3-yl-methyl]-amine

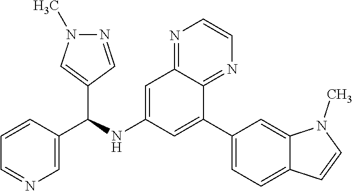 [SMILES: Cn1cc([C@@H](Nc2cc(-c3ccc4ccn(C)c4c3)c3nccnc3c2)c2cccnc2)cn1]

### Example 18: [8-(1-Methyl-1H-indol-6-yl)-quinoxalin-6-yl]-[(R)-(1-methyl-1H-pyrazol-4-yl)-pyridin-3-yl-methyl]-amine

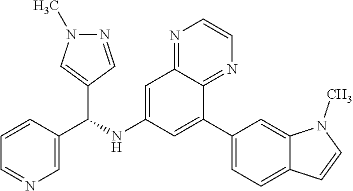 [SMILES: Cn1cc([C@H](Nc2cc(-c3ccc4ccn(C)c4c3)c3nccnc3c2)c2cccnc2)cn1]

The preparative separation of example 16 (140 mg) was performed by SFC (column: ChiralPak AD-H; eluent: CO₂:ethanol (containing 0.5% diethyl-amine) −60:40). The combined fractions were evaporated to dryness. The oily residues were dissolved in acetonitrile, diluted with water and lyophilized.

Example 17: 45 mg yellow solid; LC/MS, Rt: 1.83 min; (M+H) 446.1

Example 18: 57 mg yellow solid; LC/MS, Rt: 1.83 min; (M+H) 446.1

### Example 19: [8-(1-Methyl-1H-indol-6-yl)-quinoxalin-6-yl]-(2-methyl-1-pyridin-3-yl-propyl)-amine

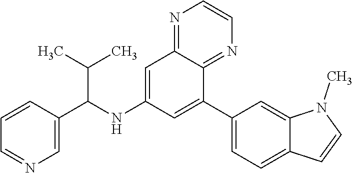 [SMILES: CC(C)C(Nc1cc(-c2ccc3ccn(C)c3c2)c2nccnc2c1)c1cccnc1]

Preparation as described for example 1 using intermediate 4 (87.0 mg; 0.291 mmol), [2-methyl-1-(3-pyridinyl)propyl]amine dihydrochloride (89.0 mg; 0.379 mmol), Tris-(dibenzylidenacetone)-dipalladium (27.2 mg; 0.029 mmol), racemic-2,2′-Bis(diphenylphosphino)-1,1′-binaphthalene (37.0 mg; 0.058 mmol) and sodium tert-butylate (113.2 mg; 1.166 mmol). Purification by flash chromatography (CombiFlashRF 200). Yield: 80.5 mg (68%) yellow foam; LC/MS, Rt: 2.01 min; (M+H) 408.2

### Chiral Separation of [8-(1-Methyl-1H-indol-6-yl)-quinoxalin-6-yl]-(2-methyl-1-pyridin-3-yl-propyl)-amine (Example 19) into Enantiomers (Conformation Arbitrarily Assigned)

### Example 20: [8-(1-Methyl-1H-indol-6-yl)-quinoxalin-6-yl]-((R)-2-methyl-1-pyridin-3-yl-propyl)-amine

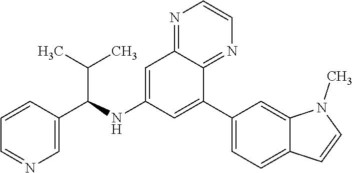 [SMILES: CC(C)[C@@H](Nc1cc(-c2ccc3ccn(C)c3c2)c2nccnc2c1)c1cccnc1]

### Example 21: [8-(1-Methyl-1H-indol-6-yl)-quinoxalin-6-yl]-((S)-2-methyl-1-pyridin-3-yl-propyl)-amine

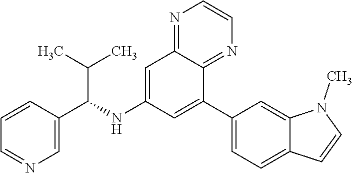 [SMILES: CC(C)[C@H](Nc1cc(-c2ccc3ccn(C)c3c2)c2nccnc2c1)c1cccnc1]

The preparative separation of example 19 (80.5 mg) was performed by SFC (column: ChiralPak AD-H; eluent: CO₂:ethanol (containing 0.5% diethyl-amine) −60:40). The combined fractions were evaporated to dryness. The oily residues were dissolved in acetonitrile, diluted with water and lyophilized.

Example 17: 38 mg yellow solid; LC/MS, Rt: 2.01 min; (M+H) 408.2

Example 18: 37 mg yellow solid; LC/MS, Rt: 2.01 min; (M+H) 408.2

### Intermediate 11

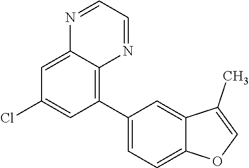 [SMILES: Cc1coc2ccc(-c3cc(Cl)cc4nccnc34)cc12]

The product was prepared by reacting 5-bromo-7-chloroquinoxaline (Intermediate 2) (360.00 mg; 1.42 mmol; 1.00 eq.), 3-methyl-5-(4,4,5,5-tetramethyl-[1,3,2]dioxaborolan-2-yl)-benzofuran* (385.65 mg; 1.42 mmol; 1.00 eq.), Cs₂CO₃ (924.92 mg; 2.84 mmol; 2.00 eq.), Pd(dppf)₂Cl₂*DCM (173.87 mg; 0.21 mmol; 0.15 eq.), DME (15.00 mL) and water (5.00 mL) in a sealed tube under argon at 120° C. for 16 h. After work-up the crude product was purified by FCC (hexane/EtOAc; gradient). 7-Chloro-5-(3-methylbenzofuran-5-yl)-quinoxaline (374.00 mg; yield 65%; 73% by UPLC) is obtained as a yellow solid.

*3-Methyl-5-(4,4,5,5-tetramethyl-[1,3,2]dioxaborolan-2-yl)-benzofuran was prepared by reacting 5-bromo-3-methylbenzofuran (150.00 mg; 0.71 mmol; 1.00 eq.), bis(pinacolato)diboron (216.57 mg; 0.85 mmol; 1.20 eq.), KOAc (209.25 mg; 2.13 mmol; 3.00 eq.), Pd(dppf)Cl₂ (52.00 mg; 0.07 mmol; 0.10 eq.) and 1,4-dioxane (4.00 mL) in a sealed tube under argon for 18 h at 100° C. and usual work-up. Purification by FCC (hexane/EtOAc: gradient). 3-Methyl-5-(4,4,5,5-tetramethyl-[1,3,2]dioxaborolan-2-yl)-benzofuran (409.00 mg; yield 73%; 83% by UPLC) was obtained as a brown solid.

### Example 22: [8-(3-Methyl-benzofuran-5-yl)-quinoxalin-6-yl]-[(1-methyl-1H-pyrazol-4-yl)-(3-methyl-3H-[1,2,3]triazol-4-yl)-methyl]-amine

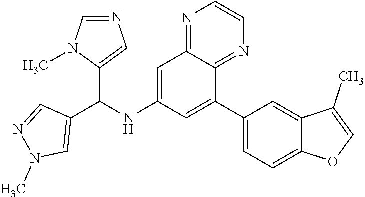 [SMILES: Cc1coc2ccc(-c3cc(NC(c4cnn(C)c4)c4cncn4C)cc4nccnc34)cc12]

Preparation as described for example 1 using intermediate 11 (125.0 mg; 0.407 mmol), intermediate 7 (117.3 mg; 0.610 mmol), Tris-(dibenzyliden-acetone)-dipalladium (37.2 mg; 0.041 mmol), racemic-2,2′-Bis(diphenylphos-phino)-1,1′-binaphthalene (50.7 mg; 0.081 mmol) and sodium tert-butylate (117.3 mg; 1.220 mmol). Purification by preparative HPLC (Agilent 1260 HPLC; column: Waters SunFire C18 5 μM 30×150 mm) and by flash chromatography (Companion RF; 24 g Si50 silica gel column). Yield: 120 mg (66%) yellow foam; LC/MS, Rt: 2.07 min; (M+H) 451.1

### Chiral Separation [8-(3-Methyl-benzofuran-5-yl)-quinoxalin-6-yl]-[(1-methyl-1H-pyrazol-4-yl)-(3-methyl-3H-[1,2,3]triazol-4-yl)-methyl]-amine (Example 22) into the Enantiomers (Conformation Arbitrarily Assigned)

### Example 23: [8-(3-Methyl-benzofuran-5-yl)-quinoxalin-6-yl]-[(S)-(1-methyl-1H-pyrazol-4-yl)-(3-methyl-3H-[1,2,3]triazol-4-yl)-methyl]-amine

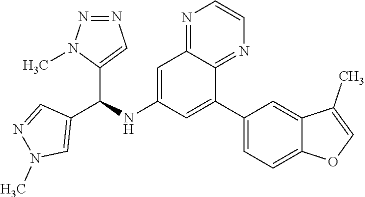 [SMILES: Cc1coc2ccc(-c3cc(N[C@@H](c4cnn(C)c4)c4cnnn4C)cc4nccnc34)cc12]

### Example 24: [8-(3-Methyl-benzofuran-5-yl)-quinoxalin-6-yl]-[(R)-(1-methyl-1H-pyrazol-4-yl)-(3-methyl-3H-[1,2,3]triazol-4-yl)-methyl]-amine

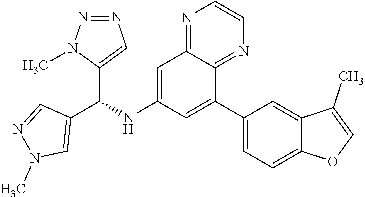 [SMILES: Cc1coc2ccc(-c3cc(N[C@H](c4cnn(C)c4)c4cnnn4C)cc4nccnc34)cc12]

The preparative separation of example 22 (120 mg) was performed by SFC (column: ChiralPak AD-H; eluent: CO₂:ethanol (containing 0.5% diethyl-amine) −60:40). The combined fractions were evaporated to dryness. The oily residues were dissolved in acetonitrile, diluted with water and lyophilized.

Example 23: 43 mg yellow solid; LC/MS, Rt: 2.07 min; (M+H) 451.2

Example 24: 38 mg yellow solid; LC/MS, Rt: 2.07 min; (M+H) 451.2

### Intermediate 12

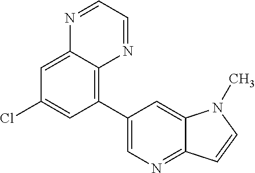 [SMILES: Cn1ccc2ncc(-c3cc(Cl)cc4nccnc34)cc21]

12.1: A reaction was charged with 5-bromo-7-chloro-quinoxaline (250.0 mg; 1.027 mmol), 6-(4,4,5,5-tetramethyl-1,3,2-dioxaborolan-2-yl)-1H-pyrrolo[3,2-b]pyridine (263.8 mg; 1.027 mmol), Pd(dppf)₂Cl₂*DCM adduct (42.8 mg; 0.051 mmol), cesium carbonate (669.7 mg; 2.053 mmol) in 1,2-dimethoxy-ethane (3.4 mL) and water (1.7 mL) and the dark red-brown suspension was stirred under argon atmosphere at 100° C. for 2 h. The reaction mixture was diluted with water and extracted twice using ethyl acetate. The combined organic layers were washed with brine, dried over sodium sulfate, filtered and concentrated in vacuo. The residue was purified by flash chromatography (CombiFlashRF 200).

Yield: 171 mg (59%) yellow solid; LC/MS, Rt: 1.39 min; (M+H) 281.0/283.0

12.2: 7-Chloro-5-(1H-pyrrolo[3,2-b]pyridin-6-yl)-quinoxaline (114.0 mg; 0.404 mmol) and cesium carbonate (292.9 mg; 0.890 mmol) were placed in a vial and suspended in DMF (3.5 mL). Iodomethane (30.8 μL; 0.485 mmol) was added and the mixture was stirred at room temperature overnight, at 50° C. for 5 h and at room temperature overnight. The reaction mixture was filtered over Celite and washed with 10 mL DMF. The filtrate was dried in vacuo and the residue purified by RP-flash chromatography (CombiFlashRF 200). The combined fractions were evaporated to dryness, the residue diluted with saturated aqueous NaHCO₃-solution and extracted with ethyl acetate. The combined organic layers were washed with brine, dried over sodium sulfate, filtered and concentrated in vacuo. Yield: 82.8 mg (68%) yellow solid; LC/MS, Rt: 1.53 min; (M+H) 295.0/297.0

### Example 25: [(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine

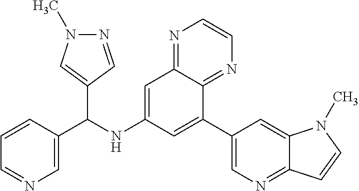 [SMILES: Cn1cc(C(Nc2cc(-c3cnc4ccn(C)c4c3)c3nccnc3c2)c2cccnc2)cn1]

Preparation as described for Example 1 using intermediate 12 (32.0 mg; 0.110 mmol), intermediate 10 (22.7 mg; 0.120 mmol), Tris-(dibenzyliden-acetone)-dipalladium (15.0 mg; 0.016 mmol), racemic-2,2′-Bis(diphenyl-phosphino)-1,1′-binaphthalene (20.5 mg; 0.033 mmol) and sodium tert-butylate (52.6 mg; 0.548 mmol). Purification by flash chromatography (CombiFlashRF 200). Yield: 36 mg (74%) yellow solid; LC/MS, Rt: 1.26 min; (M+H) 447.1

### Chiral Separation of [(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine (Example 25) (Conformation was Arbitrarily Assigned)

### Example 26: [(S)-(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine

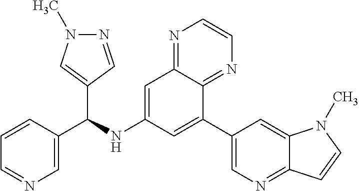 [SMILES: Cn1cc([C@@H](Nc2cc(-c3cnc4ccn(C)c4c3)c3nccnc3c2)c2cccnc2)cn1]

### Example 27: [(R)-(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine

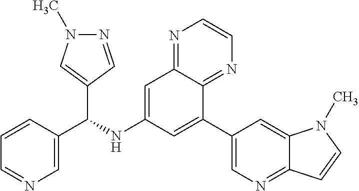 [SMILES: Cn1cc([C@H](Nc2cc(-c3cnc4ccn(C)c4c3)c3nccnc3c2)c2cccnc2)cn1]

The preparative separation of example 25 (37 mg) was performed by SFC (column: ChiralPak AS-H; eluent: CO₂:methanol (containing 0.5% diethyl-amine) −70:30). The combined fractions were evaporated to dryness. The oily residues were dissolved in acetonitrile, diluted with water and lyophilized.

Example 26: 9.8 mg yellow solid; LC/MS, Rt: 1.26 min; (M+H) 447.1; ¹H NMR (400 MHz, DMSO-d₆) δ 8.77 (d, J=2.3 Hz, 1H), 8.66-8.62 (m, 2H), 8.50-8.47 (m, 2H), 8.32-8.26 (m, 1H), 7.92 (dt, J=8.0, 2.0 Hz, 1H), 7.84-7.80 (m, 1H), 7.64 (d, J=2.5 Hz, 1H), 7.59 (s, 1H), 7.46-7.38 (m, 3H), 6.88 (d, J=2.5 Hz, 1H), 6.69-6.66 (m, 1H), 5.99 (d, J=7.1 Hz, 1H), 3.91 (s, 3H), 3.80 (s, 3H).

Example 27: 10.6 mg yellow solid; LC/MS, Rt: 1.27 min; (M+H) 447.3; ¹H NMR (400 MHz, DMSO-d₆) δ 8.78-8.75 (m, 1H), 8.66-8.60 (m, 2H), 8.49-8.47 (m, 2H), 8.30-8.21 (m, 1H), 7.93-7.89 (m, 1H), 7.83-7.76 (m, 1H), 7.64-7.61 (m, 1H), 7.58 (s, 1H), 7.46-7.38 (m, 3H), 6.88-6.86 (m, 1H), 6.69-6.64 (m, 1H), 5.98 (d, J=7.1 Hz, 1H), 3.93-3.88 (m, 3H), 3.80 (s, 3H).

### Intermediate 13

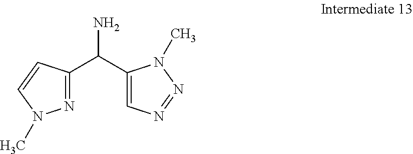

Intermediate 13 was prepared as described for intermediate 7 using 1-Methyl-1H-pyrazole-3-carbaldehyde. Yield: 1.16 g (69%) yellow solid; LC/MS, Rt: 0.39-0.48 min; (M+H) 193.1

### Example 28: [8-(1-Methyl-1H-indol-6-yl)-quinoxalin-6-yl]-[(1-methyl-1H-pyrazol-3-yl)-(3-methyl-3H-[1,2,3]triazol-4-yl)-methyl]-amine (N-[(1-methyl-1H-1,2,3-triazol-5-yl)(1-methyl-1H-pyrazol-3-yl)methyl]-8-(1-methyl-1H-indol-6-yl)quinoxalin-6-amine)

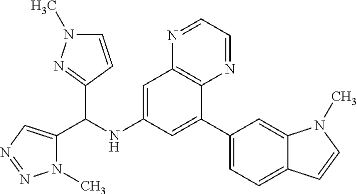 [SMILES: Cn1ccc(C(Nc2cc(-c3ccc4ccn(C)c4c3)c3nccnc3c2)c2cnnn2C)n1]

Preparation as described for Example 1 using intermediate 4 (147.5 mg; 0.502 mmol), intermediate 13 (115.9 mg; 0.603 mmol), Tris-(dibenzyliden-acetone)-dipalladium (46.0 mg; 0.050 mmol), racemic-2,2′-Bis(diphenyl-phosphino)-1,1′-binaphthalene (62.6 mg; 0.100 mmol) and sodium tert-butylate (144.9 mg; 1.507 mmol). Purification by flash chromatography (CombiFlashRF 200). Yield: 128 mg (57%) yellow foam; LC/MS, Rt: 2.03 min; (M+H) 450.2

### Chiral Separation of [8-(1-Methyl-1H-indol-6-yl)-quinoxalin-6-yl]-[(1-methyl-1H-pyrazol-3-yl)-(3-methyl-3H-[1,2,3]triazol-4-yl)-methyl]-amine (Example 28) (Conformation was Arbitrarily Assigned)

### Example 29: [8-(1-Methyl-1H-indol-6-yl)-quinoxalin-6-yl]-[(R)-(1-methyl-1H-pyrazol-3-yl)-(3-methyl-3H-[1,2,3]triazol-4-yl)-methyl]-amine (N—[(R)-(1-methyl-1H-1,2,3-triazol-5-yl)(1-methyl-1H-pyrazol-3-yl)methyl]-8-(1-methyl-1H-indol-6-yl)quinoxalin-6-amine)

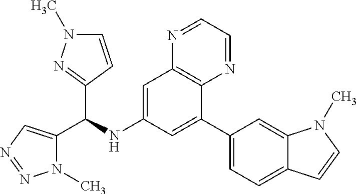 [SMILES: Cn1ccc([C@@H](Nc2cc(-c3ccc4ccn(C)c4c3)c3nccnc3c2)c2cnnn2C)n1]

### Example 30: [8-(1-Methyl-1H-indol-6-yl)-quinoxalin-6-yl]-[(S)-(1-methyl-1H-pyrazol-3-yl)-(3-methyl-3H-[1,2,3]triazol-4-yl)-methyl]-amine (N—[(S)-(1-methyl-1H-1,2,3-triazol-5-yl)(1-methyl-1H-pyrazol-3-yl)methyl]-8-(1-methyl-1H-indol-6-yl)quinoxalin-6-amine)

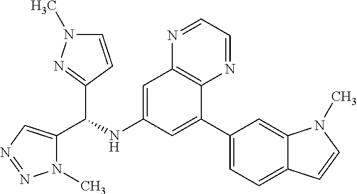 [SMILES: Cn1ccc([C@H](Nc2cc(-c3ccc4ccn(C)c4c3)c3nccnc3c2)c2cnnn2C)n1]

The preparative separation of example 28 (128 mg) was performed by SFC (column: ChiralPak AD-H; eluent: CO₂:ethanol (containing 0.5% diethyl-amine) −70:30). The combined fractions were evaporated to dryness. The oily residues were dissolved in acetonitrile, diluted with water and lyophilized.

Example 29: 53 mg yellow solid; LC/MS, Rt: 2.03 min; (M+H) 450.2; ¹H NMR (500 MHz, DMSO-d₆) δ 8.64 (d, J=1.9 Hz, 1H), 8.50 (d, J=1.9 Hz, 1H), 7.70 (d, J=2.2 Hz, 1H), 7.62-7.59 (m, 2H), 7.58-7.57 (m, 1H), 7.57 (d, J=2.6 Hz, 1H), 7.41-7.38 (m, 2H), 7.26 (dd, J=8.1, 1.5 Hz, 1H), 7.00 (d, J=2.6 Hz, 1H), 6.47 (dd, J=3.1, 0.8 Hz, 1H), 6.32 (d, J=2.2 Hz, 1H), 6.28 (d, J=7.7 Hz, 1H), 4.03 (s, 3H), 3.83 (s, 3H), 3.81 (s, 3H).

Example 30: 56 mg yellow solid; LC/MS, Rt: 2.03 min; (M+H) 450.1; ¹H NMR (500 MHz, DMSO-d₆) δ 8.64 (d, J=1.9 Hz, 1H), 8.50 (d, J=1.9 Hz, 1H), 7.70 (d, J=2.2 Hz, 1H), 7.62-7.59 (m, 2H), 7.58-7.57 (m, 1H), 7.57 (d, J=2.6 Hz, 1H), 7.41-7.38 (m, 2H), 7.26 (dd, J=8.1, 1.5 Hz, 1H), 7.00 (d, J=2.6 Hz, 1H), 6.47 (dd, J=3.1, 0.9 Hz, 1H), 6.32 (d, J=2.2 Hz, 1H), 6.28 (d, J=7.7 Hz, 1H), 4.03 (s, 3H), 3.83 (s, 3H), 3.81 (s, 3H).

The following compounds are prepared by adapting the experimental procedures herein above and choosing the appropriate starting materials:
N-[(1-methyl-1H-1,2,3-triazol-5-yl)(1-methyl-1H-pyrazol-3-yl)methyl]-8-(1-methyl-1H-indol-6-yl)quinoxalin-6-amine
N—[(S)-(1-methyl-1H-1,2,3-triazol-5-yl)(1-methyl-1H-pyrazol-3-yl)methyl]-8-(1-methyl-1H-indol-6-yl)quinoxalin-6-amine
N—[(R)-(1-methyl-1H-1,2,3-triazol-5-yl)(1-methyl-1H-pyrazol-3-yl)methyl]-8-(1-methyl-1H-indol-6-yl)quinoxalin-6-amine
8-(1-methyl-1H-indol-6-yl)-N-[(1-methyl-1H-pyrazol-4-yl)(morpholin-2-yl)methyl]quinoxalin-6-amine
8-(1-methyl-1H-indol-6-yl)-N—[(S)-(1-methyl-1H-pyrazol-4-yl)(morpholin-2-yl)methyl]]quinoxalin-6-amine
8-(1-methyl-1H-indol-6-yl)-N—[(R)-(1-methyl-1H-pyrazol-4-yl)(morpholin-2-yl)methyl]]quinoxalin-6-amine
8-(1-methyl-1H-indol-6-yl)-N-[(1-methyl-1H-pyrazol-4-yl)(pyridin-3-yl)methyl]quinoxalin-6-amine
8-(1-methyl-1H-indol-6-yl)-N—[(R)-(1-methyl-1H-pyrazol-4-yl)(pyridin-3-yl)methyl]quinoxalin-6-amine
8-(1-methyl-1H-indol-6-yl)-N—[(S)-(1-methyl-1H-pyrazol-4-yl)(pyridin-3-yl)methyl]quinoxalin-6-amine
8-(1-methyl-1H-indol-6-yl)-N-[(1-methyl-1H-pyrazol-4-yl)(4-methyl-4H-1,2,4-triazol-3-yl)methyl]quinoxalin-6-amine
8-(1-methyl-1H-indol-6-yl)-N—[(R)-(1-methyl-1H-pyrazol-4-yl)(4-methyl-4H-1,2,4-triazol-3-yl)methyl]quinoxalin-6-amine
8-(1-methyl-1H-indol-6-yl)-N—[(S)-(1-methyl-1H-pyrazol-4-yl)(4-methyl-4H-1,2,4-triazol-3-yl)methyl]quinoxalin-6-amine
8-(1-methyl-1H-indol-6-yl)-N-[(1-methyl-1H-pyrazol-3-yl)(4-methyl-4H-1,2,4-triazol-3-yl)methyl]quinoxalin-6-amine
8-(1-methyl-1H-indol-6-yl)-N—[(R)-(1-methyl-1H-pyrazol-3-yl)(4-methyl-4H-1,2,4-triazol-3-yl)methyl]quinoxalin-6-amine
8-(1-methyl-1H-indol-6-yl)-N—[(S)-(1-methyl-1H-pyrazol-3-yl)(4-methyl-4H-1,2,4-triazol-3-yl)methyl]quinoxalin-6-amine
N-[(6-methoxypyridin-3-yl)(morpholin-2-yl)methyl]-8-(1-methyl-1H-indol-6-yl)quinoxalin-6-amine
N—[(S)-(6-methoxypyridin-3-yl)(morpholin-2-yl)methyl]-8-(1-methyl-1H-indol-6-yl)quinoxalin-6-amine
N—[(R)-(6-methoxypyridin-3-yl)(morpholin-2-yl)methyl]-8-(1-methyl-1H-indol-6-yl)quinoxalin-6-amine
N-[2-(1-Methyl-1H-1,2,3-triazol-5-yl)propan-2-yl]-8-(1-methyl-1H-indol-6-yl)quinoxalin-6-amine
8-(1-Methyl-1H-indol-6-yl)-N-[2-(morpholin-2-yl)propan-2-yl]quinoxalin-6-amine.

The following compound are prepared in accordance with the procedures described in International Patent Application PCT/EP2016/000783 (published as WO 2016/180536 A1) (=Ref.):

### Comparative Example 1 (=Example 283, Compound 299 of Ref.)

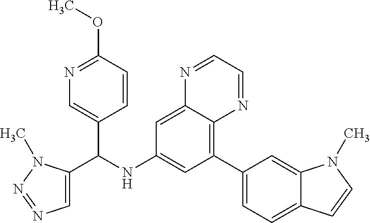 [SMILES: COc1ccc(C(Nc2cc(-c3ccc4ccn(C)c4c3)c3nccnc3c2)c2cnnn2C)cn1]

A sealed tube was charged with 7-chloro-5-(1-methyl-1H-indol-6-yl)-quinoxaline (intermediate 4) (100.00 mg; 0.34 mmol; 1.00 eq.), C-(6-methoxy-pyridin-3-yl)-C-(3-methyl-3H-[1,2,3]triazol-4-yl)-methylamine (111.95 mg; 0.51 mmol; 1.50 eq.), NaOtBu (65.43 mg; 0.68 mmol; 2.00 eq.) and toluene (5.0 mL). The reaction mixture was purged with argon and then BINAP (42.39 mg; 0.07 mmol; 0.20 eq.) and Pd₂(dba)₃ (31.17 mg; 0.03 mmol; 0.10 eq.) were added. The reaction mixture was sealed and heated at 110° C. for 16 h. After this time, the mixture was filtered through a Celite® pad and the filtrate was diluted with EtOAc and extracted with water. Combined organic phases were washed with brine, dried over Na₂SO₄. Solvent was evaporated and the residue was purified by FCC (hexane/EtOAc; gradient). (DCM:MeOH; gradient). [(6-Methoxy-pyridin-3-yl)-(3-methyl-3H-[1,2,3]triazol-4-yl)-methyl]-[8-(1-methyl-1H-indol-6-yl)-quinoxalin-6-yl]-amine (70.00 mg; yield 41.6%; 96% by HPLC) was obtained as a yellow powder.

### Comparative Examples 3 and 2 (Example 309 & Example 310, Compounds 325 & 326 of Ref.)

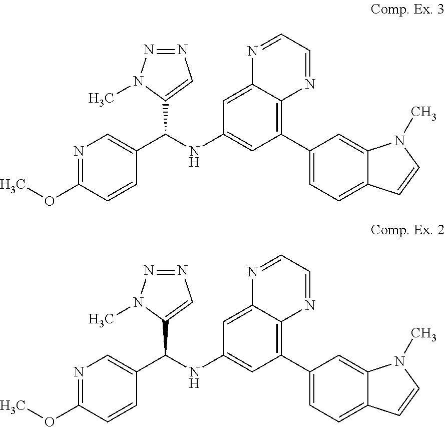 [SMILES: COc1ccc([C@@H](Nc2cc(-c3ccc4ccn(C)c4c3)c3nccnc3c2)c2cnnn2C)cn1 ; COc1ccc([C@H](Nc2cc(-c3ccc4ccn(C)c4c3)c3nccnc3c2)c2cnnn2C)cn1]

The preparative separation of the racemate [(6-methoxy-pyridin-3-yl)-(3-methyl-3H-[1,2,3]triazol-4-yl)-methyl]-[8-(1-methyl-1H-indol-6-yl)-quinoxalin-6-yl]-amine (44 mg) was performed by preparative SFC (column: ChiralPak AD-H; eluent: CO₂:iPrOH −60:40). The combined fractions were evaporated to dryness. The oily residues were dissolved in acetonitrile, diluted with water and lyophilized. N—[(R)-(6-methoxypyridin-3-yl)(1-methyl-1H-1,2,3-triazol-5-yl)methyl]-8-(1-methyl-1H-indol-6-yl)quinoxalin-6-amine (Comparative Example 3 (Example 309 of Ref.)) (18 mg; yield 44%; 99.5% by HPLC) and N—[(S)-(6-methoxypyridin-3-yl)(1-methyl-1H-1,2,3-triazol-5-yl)methyl]-8-(1-methyl-1H-indol-6-yl)quinoxalin-6-amine (Comparative Example 2 (Example 310 of Ref.)) (17 mg; yield 39%; 99% by HPLC) were obtained as yellow powders.

### Comparative Example 4 (Compound 271, Example 255 of Ref.)

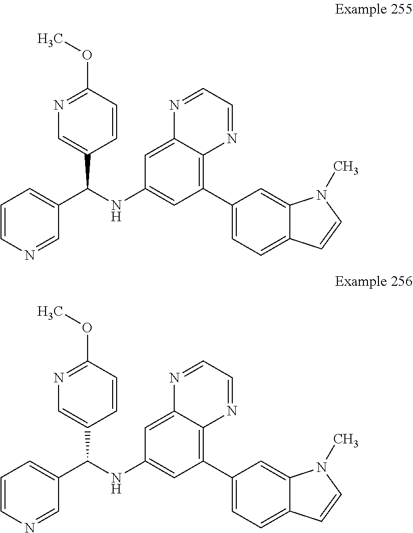 [SMILES: COc1ccc([C@@H](Nc2cc(-c3ccc4ccn(C)c4c3)c3nccnc3c2)c2cccnc2)cn1 ; COc1ccc([C@H](Nc2cc(-c3ccc4ccn(C)c4c3)c3nccnc3c2)c2cccnc2)cn1]

[(6-Methoxy-pyridin-3-yl)-pyridin-3-yl-methyl]-[8-(1-methyl-1H-indol-6-yl)-quin-oxalin-6-yl]-amine (170.000 mg; 0.356 mmol; 1.0 eq.) was dissolved in isopropyl alcohol and compound was separated by HPLC (Chiralpak AD-H; 250×20 mm I.D., 5 uM). Both enantiomers: [(R)-(6-methoxy-pyridin-3-yl)-pyridin-3-yl-methyl]-[8-(1-methyl-1H-indol-6-yl)-quinoxalin-6-yl]-amine (Example 256 of Ref.) (70.00 mg; yield 41.6%; yellow solid) and [(S)-(6-methoxy-pyridin-3-yl)-pyridin-3-yl-methyl]-[8-(1-methyl-1H-indol-6-yl)-quinoxalin-6-yl]-amine (Comparative Example 4; Example 255 of Ref.) (75.00 mg; yield 44.6%; yellow solid) are isolated with 99% of optical purity.

### Biological Activity

Biological activity of the compounds of the present invention is determined utilizing the assays described herein below.

### PFKFB3 IC50 Determination Assay

In vitro kinase assay used to determine IC₅₀ values for tested inhibitors is based on a modified ADP-Glo™ system (Promega) and consists of two parts:

1. Kinase reaction—performed in optimized conditions. At this step PFKFB3 phosphorylates its substrate fructose-6-phosphate using ATP as a source of phosphate to produce fructose-2,6-bisphosphate and ADP. Reaction is performed at Km values for ATP and substrate using optimized buffer composition and time of the reaction. Human recombinant His-tagged PFKFB3 (PFKFB3 BATCH II SEC) with confirmed activity is produced and purified in-house.
2. Detection of ADP as a product of the reaction using ADP-Glo™ system. This part is conducted by using the commercially available kit ADP-Glo™ Kinase Assay (Promega, cat. No# V9103) according to manufacturer's instruction, modified by 5× dilutions of assay reagents (both ADP-Glo™ Reagent and Kinase Detection Solution). Reproducibility and reliability of this modification is confirmed in an optimization process.

Test compounds are dissolved in DMSO and then transferred to the V-bottom 96-well plate. For IC₅₀ determination ten 10× serial dilutions starting from 100 μM are prepared.

Two mixes are prepared on ice: Mix 1—containing appropriate kinase amount in 2× reaction buffer (100 mM TRIS pH 8.0) and Mix 2—containing 2.31× concentrated substrate (Fructose-6-phosphate) and ATP in MilliQ water. 15 μL per well of Mix 1 is transferred to assay wells of 96-well white plate. Next, 2 μl of 15× concentrated test compound in DMSO is added to Mix 1 for 20 min pre-incubation, followed by addition of Mix 2 (13 μl/well). Total reaction volume is 30 μL per well. Samples are tested in duplicates. Final concentration of DMSO in the reaction is 6.7%. Conditions needed for performing PFKFB3 (PFKFB3 BATCH II SEC) in vitro kinase assay are given below:

| | Final concentration/ |
|---|---|
| Reagent/condition | final condition |
| Buffer | 100 mM Tris, pH 8.0 |
| MgCl₂ | 5 mM |
| KF | 20 mM |
| DTT | 1 mM |
| KH₂PO₄ | 5 mM |
| BSA | 0.02% |
| Tween-20 | 0.005% |
| ATP (Km) | 20 μM |
| (ultrapure, from | |
| ADP-Glo ™ kit) | |
| Substrate Fructose- | 2 μM |
| 6-phosphate (Km) | |
| Sigma cat no. | |
| F3627 | |
| In-house produced | 25 nM |
| human recombinant | |
| PFKFB3 (PFKFB3 | |
| BATCH II SEC) | |
| Time of reaction | 2 h |
| Temperature of | rt |
| reaction | |

This protocol is based on Technical Bulletin, ADP-Glo™ Kinase Assay (Promega) and is adapted to 96-well plate containing 30 μL reaction mixture:

30 μL of 5× diluted ADP-Glo™ Reagent is added to each well of 96-well plate containing 30 μL of reaction mixture. The plate is incubated for 90 minutes on a shaker at rt. 60 μL of 5× diluted Kinase Detection Solution is added to each well of 96-well plate containing 60 μL of the solution (ratio of kinase reaction volume to ADP-Glo™ Reagent volume to Kinase Detection Solution volume is maintained at 1:1:2). Plate is incubated for 40 minutes on a shaker at rt, protected from light. Luminescence is measured in the plate reader Synergy 2 (BioTek).

Luminescent readouts for compounds tested in 10 concentrations (routinely from 100 μM to 1 nM, 10-fold serial dilutions) in duplicates, as well as for positive control, are first normalized to no-substrate negative control by its subtraction. In the next step, % of normalized positive control is calculated for each data point and plotted against test compound concentration:

$$\% of control = 100 \% \times \frac{( {Lum}_{cpd} - {Lum}_{neg} )}{( {Lum}_{pos} - {Lum}_{neg} )}$$

% of control—percent of positive control normalized to no-substrate negative control
Lum_{cpd}—luminescence of test compound
Lum_{neg}—luminescence of no-substrate negative control
Lumₚₒₛ—luminescence of positive control

IC₅₀ parameter is determined by the GraphPad Prism 5.0 software [log(inhibitor) vs. response—Variable slope (four parameters)].

IC₅₀ values of compounds of the present invention and comparative examples are shown in Table 1 below.

### Test Method Microsomal Stability (Intrinsic Clearance)

A microsomal stability assay is used to measure in vitro clearance (Clint). The assay involves measuring the rate of disappearance of a compound due to its intrinsic attitude to be metabolized (“intrinsic” meaning that the disappearance is not affected by other properties like permeability, binding etc. that play a role when quantifying in vivo clearance). The microsomal stability (intrinsic clearance, Clint) and thus metabolic stability is generally given as μl/min/mg protein. It can be visualized as the volume of solution that 1 mg of microsomes is able to clear of the compound in one minute.

### Instrumentation

A Tecan Genesis workstation (RSP 150/8) was used for to perform the microsomal incubations. Analysis was carried out using a Waters ACQUITY UPLC system coupled to an ABSciex API3000 mass spectrometer. Data analysis was performed using Assay Explorer (Symyx).

### UPLC Conditions

### Column: Acquity UPLC BEH C18, 2.1×50 mm, 1.7 μm (Waters)

Mobile phases: A=0.1% formic acid in water; B=acetonitrile

| Gradient Time | % A | % B |
|---|---|---|
| initial | 90 | 10 |
| 0.47 | 5 | 95 |
| 0.65 | 5 | 95 |
| 0.66 | 90 | 10 |

Flow rate: 0.750 mL/min; Detection: ESI, MRM; Injection: 10 μL; Column temperature: 50° C.

### Chemicals

Potassium phosphate buffer: 0.05 M potassium phosphate buffer pH 7.4 containing 1 mM MgCl₂
NADPH (nicotinamide adenine dinucleotide phosphate): 22.5 mg NADPH-Na₄ in 1.8 ml potassium phosphate buffer
Acetonitrile: 50 Vol % acetonitrile (1 volume acetonitrile, 1 volume water)
DMSO: 20 Vol % DMSO in water
Stock solution of 20 mg/ml human or mouse liver microsomes (protein)/ml in phosphate buffer
Stock solution of 10 mM compound in 100% DMSO

Values of microsomal stability of compounds of the present invention as well as comparative examples are shown in Table 1 below.

### Test Method Kinetic Solubility

Kinetic solubility of compounds of the present invention were tested according the following procedure. Test results are given in Table 1 below.

### Reagents and Materials:

(a) Buffer Solution Phosphate Buffer 20 mM pH 7.4 is prepared using Sorensen Phosphate Buffer 0.2 M pH 7.4 (EMS) in a 1000 mL volumetric flask
(b) 2% DMSO in Phosphate Buffer 20 mM pH 7.4 is prepared by using 1 mL Dimethylsulfoxide (Merck) and 49 ml Sorensen Phosphate Buffer 20 mM pH 7.4
(c) Acetonitrile/Methanole/Eluent A (1:1:2; v/v/v) is prepared by using 50 mL Acetonitrile (Merck), 50 mL Methanol (Merck) and 100 mL Eluent A
(d) Eluent A is prepared by using 1 mL Formic Acid (Merck) and 999 mL Ultrapure water
(e) Eluent B is prepared by using 1 mL Formic Acid (Merck) and 999 mL Acetonitrile (Merck)
(f) Filtration plate: Millipore MultiScreen HTS HV (0.45 μm) 96 Well (Millipore)

### (g) PP-Plate: Microplate, 96 Well, V-Shape, (Greiner)

### (h) Column: Waters XBridge Column C8 3.5 μm (Waters)

(i) HPLC Vials: 1.5 mL short thread vial, 32×11.6 mm, clear glass, 1st hydrolytic class, wide opening (VWR)
(j) Micro-Inserts: 0.1 mL micro-insert, 31×6 mm, clear glass, 15 mm top (VWR)
(k) Screw-caps: 9 mm combination seal: PP short thread cap, black, centre hole silicone white/PTFE blue, 55° shore A, 1.0 mm, slitted (VWR)

### Preparation of the Sample:

98 μL buffer solution (a) is added into a well of a 96 well filtration plate (f). Then 2 μL of 10 mM test compound solution in DMSO (Remp tube) is added. The filtration plate is incubated at room temperature for 120 minutes while agitating at 250 rpm and centrifuged with the PP-Plate (g) as receiver plate below (2500 rpm, 3 min). The 50 μL of filtrate is taken from the receiver plate and mixed with 50 μL of 2% DMSO in buffer pH 7.4 solution (b) (dilution factor=2) in a HPLC Vial (i)+(j).

### Preparation of the Standard:

Standard is prepared by diluting 2 μL of the same 10 mM test compound solution (Remp tube) diluted with 198 μL Acetonitrile/Methanole/Eluent A (1:1:2; v/v/v) (c) to prepare a 100 μM standard solution in a HPLC Vial (i)+(j).

### Chromatographic Conditions:

### Column: Waters XBridge Column C8 3.5 μm (Waters)

Column temperature: 37° C.
Autosampler: room temperature (approx. 25° C.)
Gradient programme:

| Time [min] | Eluent A (d) [%] | Eluent B (e) [%] | flow [mL/min] |
|---|---|---|---|
| 0.0 | 90 | 10 | 1.7 |
| 0.3 | 90 | 10 | 1.7 |
| 2.0 | 10 | 90 | 1.7 |
| 2.75 | 10 | 90 | 1.7 |
| 2.76 | 90 | 10 | 2.5 |
| 4.0 | 90 | 10 | 2.5 |

Injection volume: 10 μL
Wavelength: Suitable wavelength (maximum sensitivity) selected out of wavelength range of 190-400 nm using a DAD detector.

L [μg/mL]=(area sample*concentration standard*dilution factor sample)/(area standard*dilution factor standard)

S [mol/L]=(L [μg/mL]*molar weight [g/mol])/1000 Calculation:

(S: kinetic solubility)

### Solubility PBS

Thermodynamic solubility in PBS at pH 7.4 of test compounds is determined using the shake flask method and HPLC as described below. Test results are shown in Table 1 below.

The method involves dissolving the test compound in a solvent at a constant temperature followed by HPLC determination of the concentration of the solute in the solution, which must not contain any undissolved particles.

### Procedure:

Phosphate Buffer pH 7.4 is prepared by using 50 mL monobasic potassium phosphate solution 0.2 M in a 200-mL volumetric flask and adding 39.1 mL of sodium hydroxide solution 0.2 M and then adding water to volume.

Standard solution is prepared by weighing in a standard compound (about 1 mg) into a flask and dissolved completely in a solution of acetonitrile/methanol (1:1; V/V).

The sample of the test compound (about 2-3 mg) is weighed into a Uniprep® syringeless filter (5 mL; 0.45 μm), the 2 mL of solvent is added and the mixture is agitated for 24 hours at 37° C. The suspension is filtered after 24 hours and the concentration of dissolved substance is determined by HPLC. The result is stated as >x μg/mL, calculated from the sample weight taken and the volume of solvent used

### Chromatographic Conditions:

### Solvent System:

Eluent A: Ultrapure water/Formic acid GR for analysis (999:1, V/V)
Eluent B: Acetonitrile/Formic acid GR for analysis (999:1, V/V)

### Equipment Settings:

Wavelength: Suitable wavelength in the range of 190-400 nm.

### Column: Chromolith RP18e 100×3 mm

Oven temperature: 37° C.

### Auto-Sampler: 37° C.

Column thermostat: 37° C.
Gradient program:

| Time [Min.] | Eluent A [%] | Eluent B [%] | Flow [mL/min] |
|---|---|---|---|
| 0 | 90 | 10 | 0.85 |
| 0.6 | 90 | 10 | 0.85 |
| 4 | 10 | 90 | 0.85 |
| 5.5 | 10 | 90 | 0.85 |
| 5.51 | 90 | 10 | 2.50 |
| 8 | 90 | 10 | 2.50 |

The result is determined quantitatively based on the external standard method through integration of the peak areas with reference to the figures obtained for the standard substance.

L[μg/mL]=a(A)*c(S)*F(A)/a(S)*F(S) Calculation:
a (A)=peak area for analyte/mL
a (S)=peak area for standard/mL
c (S)=concentration of standard (μg/mL)
F (A)=dilution factor for analyte
F (S)=dilution factor for standard

TABLE 1

| | | | | | CLᵢₙₜ |
|---|---|---|---|---|---|
| Example No./ | PFKFB | Kinetic | Solubility PBS/ | CLᵢₙₜ Mouse | Human |
| Comparative | IC50 | solubility | pH 7.4 | [μl/min/mg | [μl/min/mg |
| Example No. | [nM] | [mmol/l] | [mg/mL] | prot] | prot] |
| Comparative | 25 | | | 188 | 163 |
| Example 1 (=Cpd. | | | | | |
| 299, Ex. 283 of Ref.) | | | | | |
| Comparative | 3.5 | 0.022 | | 134 | 113 |
| Example 2 (=Cpd. | | | | | |
| 326, Ex. 310 of Ref.) | | | | | |
| Comparative | 530 | 0.022 | | 287 | 267 |
| Example 3 (=Cpd. | | | | | |
| 325, Ex. 309 of Ref.) | | | | | |
| Comparative | 9.9 | 0.0037 | 0.001 | 291 | 214 |
| Example 4 (=Cpd. | | | | | |
| 271, Ex. 255) | | | | | |
| Example 1 | 32 | 0.069 | | 122 | 78 |
| Example 2 | 28 | 0.061 | 0.01 | 101 | 78 |
| Example 3 | 180 | 0.064 | | 92 | 61 |
| Example 5 | 8 | 0.02 | 0.004 | 154 | 264 |
| Example 6 | 900 | 0.018 | | 134 | 174 |
| Example 8 | 5.2 | 0.11 | 0.08 | 29 | 50 |
| Example 9 | 87 | 0.11 | | 146 | 52 |
| Example 11 | 14 | 0.10 | 0.004 | 105 | 130 |
| Example 12 | 44 | | | | |
| Example 14 | 640 | 0.036 | | 67 | 57 |
| Example 15 | 560 | 0.048 | | 88 | 76 |
| Example 17 | 120 | 0.042 | | 95 | 172 |
| Example 18 | 6.5 | 0.039 | | 148 | 142 |
| Example 20 | 33 | 0.006 | | >500 | >500 |
| Example 21 | 23 | 0.007 | | 488 | 366 |
| Example 23 | 4.2 | 0.085 | | 99 | 78 |
| Example 24 | 35 | 0.083 | | 374 | 85 |
| Example 26 | 1100 | 0.192 | | 58 | 67 |
| Example 27 | 6300 | 0.161 | | 64 | 404 |
| Example 29 | 22 | 0.108 | | 69 | 51 |
| Example 30 | 190 | 0.096 | | 216 | 132 |

Comparative Examples as described in Ref.=International Patent Application PCT/EP2016/000783 published as WO 2016/180536 A1).

The following examples relate to medicaments:

### Example A: Injection Vials

A solution of 100 g of an active compound of the formula I and 5 g of diso-dium hydrogenphosphate in 3 l of bidistilled water is adjusted to pH 6.5 using 2 N hydrochloric acid, sterile filtered, transferred into injection vials, lyophilised under sterile conditions and sealed under sterile conditions. Each injection vial contains 5 mg of active compound.

### Example B: Suppositories

A mixture of 20 g of an active compound of the formula I with 100 g of soya lecithin and 1400 g of cocoa butter is melted, poured into moulds and allowed to cool. Each suppository contains 20 mg of active compound.

### Example C: Solution

A solution is prepared from 1 g of an active compound of the formula I, 9.38 g of NaH₂PO₄.2H₂O, 28.48 g of Na₂HPO₄.12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example D: Ointment

500 mg of an active compound of the formula I are mixed with 99.5 g of Vaseline under aseptic conditions.

### Example E: Tablets

A mixture of 1 kg of active compound of the formula I, 4 kg of lactose, 1.2 kg of potato starch, 0.2 kg of talc and 0.1 kg of magnesium stearate is pressed in a conventional manner to give tablets in such a way that each tablet contains 10 mg of active compound.

### Example F: Dragees

Tablets are pressed analogously to Example E and subsequently coated in a conventional manner with a coating of sucrose, potato starch, talc, tragacanth and dye.

### Example G: Capsules

2 kg of active compound of the formula I are introduced into hard gelatine capsules in a conventional manner in such a way that each capsule contains 20 mg of the active compound.

### Example H: Ampoules

A solution of 1 kg of active compound of the formula I in 60 l of bidistilled water is sterile filtered, transferred into ampoules, lyophilised under sterile conditions and sealed under sterile conditions. Each ampoule contains 10 mg of active compound.

1: A compound of formula I 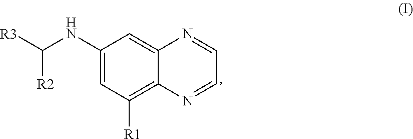
wherein
R1 denotes N-methyl-indol-6-yl (1-methyl-1H-indol-6-yl), 3-methyl-1-benzofuran-5-yl, or 1-methyl-1H-pyrrolo[3,2-b]pyridin-6-yl;
R2 denotes 1H-pyrazol-4-yl or 1-methyl-1H-pyrazol-4-yl; and
R3 denotes 1H-imidazol-2-yl, 1-methyl-1H-imidazol-2-yl, 1H-imidazol-5-yl, 1-methyl-1H-imidazol-5-yl, 1H-1,2,3-triazol-5-yl, 1-methyl-1H-1,2,3-triazol-5-yl, morpholin-2-yl, morpholin-3-yl, pyridin-3-yl, pyridin-4-yl, 4H-1,2,4-triazol-3-yl, or 4-methyl-4H-1,2,4-triazol-3-yl;
or
R2 denotes 1H-pyrazol-3-yl or 1-methyl-1H-pyrazol-3-yl; and
R3 denotes 1H-1,2,3-triazol-5-yl, 1-methyl-1H-1,2,3-triazol-5-yl, 4H-1,2,4-triazol-3-yl, or 4-methyl-4H-1,2,4-triazol-3-yl;
or
R2 denotes 1H-pyridazin-6-on-3-yl or 6-methoxypyridazin-3-yl; and
R3 denotes pyridin-3-yl or pyridin-4-yl;
or derivatives, N-oxides, prodrugs, solvates, tautomers or stereoisomers thereof as well as the physiologically acceptable salts of each of the foregoing, including mixtures thereof in all ratios.

2: The compound according to claim 1, or derivatives, N-oxides, prodrugs, solvates, tautomers or stereoisomers thereof as well as the physiologically acceptable salts of each of the foregoing, including mixtures thereof in all ratios,
wherein
R1 denotes N-methyl-indol-6-yl (1-methyl-1H-indol-6-yl) or 3-methyl-1-benzofuran-5-yl;
R2 denotes 1-methyl-1H-pyrazol-4-yl; and
R3 denotes 1-methyl-1H-imidazol-2-yl, 1-methyl-1H-imidazol-5-yl, 1-methyl-1H-1,2,3-triazol-5-yl, morpholin-2-yl, pyridine-3-yl, or 4-methyl-4H-1,2,4-triazol-3-yl;
or
R2 denotes 1-methyl-1H-pyrazol-3-yl; and
R3 denotes 1-methyl-1H-1,2,3-triazol-5-yl or 4-methyl-4H-1,2,4-triazol-3-yl;
or
R2 denotes 1H-pyridazin-6-on-3-yl or 6-methoxypyridazin-3-yl; and
R3 denotes pyridine-3-yl.

3: The compound according to claim 1, or derivatives, N-oxides, prodrugs, solvates, tautomers or stereoisomers thereof as well as the physiologically acceptable salts of each of the foregoing, including mixtures thereof in all ratios,
wherein
R1 denotes N-methyl-indol-6-yl (1-methyl-1H-indol-6-yl) or 3-methyl-1-benzofuran-5-yl;
R2 denotes 1-methyl-1H-pyrazol-4-yl; and
R3 denotes 1-methyl-1H-imidazol-2-yl, 1-methyl-1H-imidazol-5-yl, 1-methyl-1H-1,2,3-triazol-5-yl, morpholin-2-yl, pyridine-3-yl, or 4-methyl-4H-1,2,4-triazol-3-yl.

4: The compound according to claim 1, or derivatives, N-oxides, prodrugs, solvates, tautomers or stereoisomers thereof as well as the physiologically acceptable salts of each of the foregoing, including mixtures thereof in all ratios,
wherein
R1 denotes N-methyl-indol-6-yl (1-methyl-1H-indol-6-yl) or 3-methyl-1-benzofuran-5-yl;
R2 denotes 1-methyl-1H-pyrazol-4-yl; and
R3 denotes 1-methyl-1H-imidazol-2-yl, 1-methyl-1H-imidazol-5-yl, or 1-methyl-1H-1,2,3-triazol-5-yl.

5: The compound according to claim 1, or derivatives, N-oxides, prodrugs, solvates, tautomers or stereoisomers thereof as well as the physiologically acceptable salts of each of the foregoing, including mixtures thereof in all ratios,
wherein
R1 denotes N-methyl-indol-6-yl (1-methyl-1H-indol-6-yl) or 3-methyl-1-benzofuran-5-yl;
R2 denotes 1-methyl-1H-pyrazol-4-yl; and
R3 denotes 1-methyl-1H-1,2,3-triazol-5-yl.

6: The compound according to claim 1, or derivatives, N-oxides, prodrugs, solvates, tautomers or stereoisomers thereof as well as the physiologically acceptable salts of each of the foregoing, including mixtures thereof in all ratios,
wherein
R1 denotes N-methyl-indol-6-yl;
R2 denotes 1-methyl-1H-pyrazol-3-yl; and
R3 denotes 1-methyl-1H-1,2,3-triazol-5-yl or 4-methyl-4H-1,2,4-triazol-3-yl.

7: The compound according to claim 1, or derivatives, N-oxides, prodrugs, solvates, tautomers or stereoisomers thereof as well as the physiologically acceptable salts of each of the foregoing, including mixtures thereof in all ratios,
wherein
R1 denotes N-methyl-indol-6-yl;
R2 denotes 1H-pyridazin-6-on-3-yl or 6-methoxypyridazin-3-yl; and
R3 denotes pyridin-3-yl.

8: A compound, or the N-oxides and/or physiologically acceptable salts thereof, wherein the compound is at least one member selected from the group consisting of:
6-[{[8-(1-methyl-1H-indol-6-yl)quinoxalin-6-yl]amino}(pyridin-3-yl)methyl]-2,3-dihydropyridazin-3-one (6-{[8-(1-Methyl-1H-indol-6-yl)-quinoxalin-6-ylamino]-pyridin-3-yl-methyl}-2H-pyridazin-3-one),
6-[(S)-{[8(1-methyl-1H-indol-6-yl)quinoxalin-6-yl]amino}(pyridin-3-yl)methyl]-2,3-dihydropyridazin-3-one,
6-[(R)-{[8-(1-methyl-1H-indol-6-yl)quinoxalin-6-yl]amino}(pyridin-3-yl)methyl]-2,3-dihydropyridazin-3-one,
N-[(1-methyl-1H-imidazol-2-yl)(1-methyl-1H-pyrazol-4-yl)methyl]-8-(1-methyl-1H-indol-6-yl)quinoxalin-6-amine,
N—[(S)-(1-methyl-1H-imidazol-2-yl)(1-methyl-1H-pyrazol-4-yl)methyl]-8-(1-methyl-1H-indol-6-yl)quinoxalin-6-amine,
N—[(R)-(1-methyl-1H-imidazol-2-yl)(1-methyl-1H-pyrazol-4-yl)methyl]-8-(1-methyl-1H-indol-6-yl)quinoxalin-6-amine,
N-[(6-methoxypyridazin-3-yl)(pyridin-3-yl)methyl]-8-(1-methyl-1H-indol-6-yl)quinoxalin-6-amine,
N—[(S)-(6-methoxypyridazin-3-yl)(pyridin-3-yl)methyl]-8-(1-methyl-1H-indol-6-yl)quinoxalin-6-amine,
N—[(R)-(6-methoxypyridazin-3-yl)pyridin-3-yl)methyl]-8-(1-methyl-1H-indol-6-yl)quinoxalin-6-amine,
N-[(1-methyl-1H-1,2,3-triazol-5-yl)(1-methyl-1H-pyrazol-4-yl)methyl]-8-(1-methyl-1H-indol-6-yl)quinoxalin-6-amine ([8-(1-Methyl-1H-indol-6-yl)-quinoxalin-6-yl]-[(1-methyl-1H-pyrazol-4-yl)-(3-methyl-3H-[1,2,3]triazol-4-yl)-methyl]-amine),
N—[(S)-(1-methyl-1H-1,2,3-triazol-5-yl)(1-methyl-1H-pyrazol-4-yl)methyl]-8-(1-methyl-1H-indol-6-yl)quinoxalin-6-amine,
N—[(R)-(1-methyl-1H-1,2,3-triazol-5-yl)(1-methyl-1H-pyrazol-4-yl)methyl]-8-(1-methyl-1H-indol-6-yl)quinoxalin-6-amine,
N-(1-methyl-1H-imidazol-5-yl)(1-methyl-1H-pyrazol-4-yl)methyl]-8-(1-methyl-1H-indol-6-yl)quinoxalin-6-amine ([(3-Methyl-3H-imidazol-4-yl)-(1-methyl-1H-pyrazol-4-yl)-methyl]-[8-(1-methyl-1H-indol-6-yl)-quinoxalin-6-yl]-amine),
N—[(S)-(1-methyl-1H-imidazol-5-yl)(1-methyl-1H-pyrazol-4-yl)methyl]-8-(1-methyl-1H-indol-6-yl)quinoxalin-6-amine,
N—[(R)-(1-methyl-1H-imidazol-5-yl)(1-methyl-1H-pyrazol-4-yl)methyl]-8-(1-methyl-1H-indol-6-yl)quinoxalin-6-amine,
N-[(1-methyl-1H-1,2,3-triazol-5-yl)(1-methyl-1H-pyrazol-3-yl)methyl]-8-(1-methyl-1H-indol-6-yl)quinoxalin-6-amine ([8-(1-Methyl-1H-indol-6-yl)-quinoxalin-6-yl]-[(1-methyl-1H-pyrazol-3-yl)-(3-methyl-3H-[1,2,3]triazol-4-yl)-methyl]-amine),
N—[(S)-(1-methyl-1H-1,2,3-triazol-5-yl)(1-methyl-1H-pyrazol-3-yl)methyl]-8-(1-methyl-1H-indol-6-yl)quinoxalin-6-amine,
N—[(R)-(1-methyl-1H-1,2,3-triazol-5-yl)(1-methyl-1H-pyrazol-3-yl)methyl]-8-(1-methyl-1H-indol-6-yl)quinoxalin-6-amine,
8-(1-methyl-1H-indol-6-yl)-N-[(1-methyl-1H-pyrazol-4-yl)(morpholin-2-yl)methyl]quinoxalin-6-amine,
8-(1-methyl-1H-indol-6-yl)-N—[(S)-(1-methyl-1H-pyrazol-4-yl)(morpholin-2-yl)methyl]]quinoxalin-6-amine,
8-(1-methyl-1H-indol-6-yl)-N—[(R)-(1-methyl-1H-pyrazol-4-yl)(morpholin-2-yl)methyl]quinoxalin-6-amine,
8-(1-methyl-1H-indol-6-yl)-N-[(1-methyl-1H-pyrazol-4-yl)(pyridin-3-yl)methyl]quinoxalin-6-amine,
8-(1-methyl-1H-indol-6-yl)-N—[(R)-(1-methyl-1H-pyrazol-4-yl)(pyridin-3-yl)methyl]quinoxalin-6-amine,
8-(1-methyl-1H-indol-6-yl)-N—[(S)-(1-methyl-1H-pyrazol-4-yl)(pyridin-3-yl)methyl]quinoxalin-6-amine,
8-(1-methyl-1H-indol-6-yl)-N-[(1-methyl-1H-pyrazol-4-yl)(4-methyl-4H-1,2,4-triazol-3-yl)methyl]quinoxalin-6-amine,
8-(1-methyl-1H-indol-6-yl)-N—[(S)-(1-methyl-1H-pyrazol-4-yl)(4-methyl-4H-1,2,4-triazol-3-yl)methyl]quinoxalin-6-amine,
8-(1-methyl-1H-indol-6-yl)-N—[(S)-(1-methyl-1H-pyrazol-4-yl)(4-methyl-4H-1,2,4-triazol-3-yl)methyl]quinoxalin-6-amine,
8-(1-methyl-1H-indol-6-yl)-N-[(1-methyl-1H-pyrazol-3-yl)(4-methyl-4H-1,2,4-triazol-3-yl)methyl]quinoxalin-6-amine,
8-(1-methyl-1H-indol-6-yl)-N—[(S)-(1-methyl-1H-pyrazol-3-yl)(4-methyl-4H-1,2,4-triazol-3-yl)methyl]quinoxalin-6-amine,
8-(1-methyl-1H-indol-6-yl)-N—[(S)-(1-methyl-1H-pyrazol-3-yl)(4-methyl-4H-1,2,4-triazol-3-yl)methyl]quinoxalin-6-amine,
8-(3-methyl-1-benzofuran-5-yl)-N-[(1-methyl-1H-1,2,3-triazol-5-yl)(1-methyl-1H-pyrazol-4-yl)methyl]quinoxalin-6-amine ([8-(3-Methyl-benzofuran-5-yl)-quinoxalin-6-yl]-[(1-methyl-1H-pyrazol-4-yl)-(3-methyl-3H-[1,2,3]triazol-4-yl)-methyl]-amine),
8-(3-methyl-1-benzofuran-5-yl)-N—[(S)-(1-methyl-1H-1,2,3-triazol-5-yl)(1-methyl-1H-pyrazol-4-yl)methyl]quinoxalin-6-amine,
8-(3-methyl-1-benzofuran-5-yl)-N—[(S)-(1-methyl-1H-1,2,3-triazol-5-yl)(1-methyl-1H-pyrazol-4-yl)methyl]quinoxalin-6-amine,
N-[(6-methoxypyridin-3-yl)(morpholin-2-yl)methyl]-8-(1-methyl-1H-indol-6-yl)quinoxalin-6-amine,
N—[(S)-(6-methoxypyridin-3-yl)(morpholin-2-yl)methyl]-8-(1-methyl-1H-indol-6-yl)quinoxalin-6-amine,
N—[(R)-(6-methoxypyridin-3-yl)(morpholin-2-yl)methyl]-8-(1-methyl-1H-indol-6-yl)quinoxalin-6-amine,
[8-(1-Methyl-1H-indol-6-yl)-quinoxalin-6-yl]-(2-methyl-1-pyridin-3-yl-propyl)-amine,
[8-(1-Methyl-1H-indol-6-yl)-quinoxalin-6-yl]-((R)-2-methyl-1-pyridin-3-yl-propyl)-amine,
[8-(1-Methyl-1H-indol-6-yl)-quinoxalin-6-yl]-((S)-2-methyl-1-pyridin-3-yl-propyl)-amine,
N-[2-(1-Methyl-1H-1,2,3-triazol-5-yl)propan-2-yl]-8-(1-methyl-1H-indol-6-yl)quinoxalin-6-amine,
8-(1-Methyl-1H-indol-6-yl)-N-[2-(morpholin-2-yl)propan-2-yl]quinoxalin-6-amine,
[(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine,
[(S)-(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine, and
[(R)-(1-Methyl-1H-pyrazol-4-yl)-pyridin-3-yl-methyl]-[8-(1-methyl-1H-pyrrolo[3,2-b]pyridin-6-yl)-quinoxalin-6-yl]-amine.

9: The compound according to claim 8, or the N-oxides and/or physiologically acceptable salts thereof, wherein the compound is at least one member selected from the group consisting of:
6-[{[8-(1-methyl-1H-indol-6-yl)quinoxalin-6-yl]amino}(pyridin-3-yl)methyl]-2,3-dihydropyridazin-3-one,
6-[(S)-{[8-(1-methyl-1H-indol-6-yl)quinoxalin-6-yl]amino}(pyridin-3-yl)methyl]-2,3-dihydropyridazin-3-one,
6-[(R)-{[8-(1-methyl-1H-indol-6-yl)quinoxalin-6-yl]amino}(pyridin-3-yl)methyl]-2,3-dihydropyridazin-3-one,
N—[(S)-(1-methyl-1H-imidazol-2-yl)(1-methyl-1H-pyrazol-4-yl)methyl]-8-(1-methyl-1H-indol-6-yl)quinoxalin-6-amine,
N—[(R)-(1-methyl-1H-imidazol-2-yl)(1-methyl-1H-pyrazol-4-yl)methyl]-8-(1-methyl-1H-indol-6-yl)quinoxalin-6-amine,
N—[(S)-(6-methoxypyridazin-3-yl)(pyridin-3-yl)methyl]-8-(1-methyl-1H-indol-6-yl)quinoxalin-6-amine,
N—[(R)-(6-methoxypyridazin-3-yl)(pyridin-3-yl)methyl]-8-(1-methyl-1H-indol-6-yl)quinoxalin-6-amine,
N—[(S)-(1-methyl-1H-1,2,3-triazol-5-yl)(1-methyl-1H-pyrazol-4-yl)methyl]-8-(1-methyl-1H-indol-6-yl)quinoxalin-6-amine,
N—[(R)-(1-methyl-1H-1,2,3-triazol-5-yl)(1-methyl-1H-pyrazol-4-yl)methyl]-8-(1-methyl-1H-indol-6-yl)quinoxalin-6-amine,
N—[(S)-(1-methyl-1H-imidazol-5-yl)(1-methyl-1H-pyrazol-4-yl)methyl]-8-(1-methyl-1H-indol-6-yl)quinoxalin-6-amine,
N—[(R)-(1-methyl-1H-imidazol-5-yl)(1-methyl-1H-pyrazol-4-yl)methyl]-8-(1-methyl-1H-indol-6-yl)quinoxalin-6-amine,
8-(1-methyl-1H-indol-6-yl)-N—[(R)-(1-methyl-1H-pyrazol-4-yl)(pyridin-3-yl)methyl]quinoxalin-6-amine,
8-(1-methyl-1H-indol-6-yl)-N—[(S)-(1-methyl-1H-pyrazol-4-yl)(pyridin-3-yl)methyl]quinoxalin-6-amine,
8-(3-methyl-1-benzofuran-5-yl)-N—[(S)-(1-methyl-1H-1,2,3-triazol-5-yl)(1-methyl-1H-pyrazol-4-yl)methyl]quinoxalin-6-amine,
8-(3-methyl-1-benzofuran-5-yl)-N—[(S)-(1-methyl-1H-1,2,3-triazol-5-yl)(1-methyl-1H-pyrazol-4-yl)methyl]quinoxalin-6-amine,
N—[(S)-(1-methyl-1H-1,2,3-triazol-5-yl)(1-methyl-1H-pyrazol-3-yl)methyl]-8-(1-methyl-1H-indol-6-yl)quinoxalin-6-amine, and
N—[(R)-(1-methyl-1H-1,2,3-triazol-5-yl)(1-methyl-1H-pyrazol-3-yl)methyl]-8-(1-methyl-1H-indol-6-yl)quinoxalin-6-amine.

10: A pharmaceutical composition, comprising:
at least one compound according to claim 1, or its derivatives, N-oxides, prodrugs, solvates, tautomers or stereoisomers thereof as well as the physiologically acceptable salts of each of the foregoing, including mixtures thereof in all ratios, as active ingredient, and
a pharmaceutically acceptable carrier.

11: The pharmaceutical composition according to claim 10, further comprising a second active ingredient or its derivatives, N-oxides, prodrugs, solvates, tautomers or stereoisomers thereof as well as the physiologically acceptable salts of each of the foregoing, including mixtures thereof in all ratios,
wherein that second active ingredient is other than a compound according to claim 1.

12: A medicament, comprising:
at least one compound according to claim 1, or its derivatives, N-oxides, prodrugs, solvates, tautomers or stereoisomers thereof as well as the physiologically acceptable salts of each of the foregoing, including mixtures thereof in all ratios.

13: (canceled)

14: (canceled)

15: A kit, comprising:
separate packs of
a) an effective amount of the compound according to claim 1, or its derivatives, N-oxides, prodrugs, solvates, tautomers or stereoisomers thereof as well as the physiologically acceptable salts of each of the foregoing, including mixtures thereof in all ratios; and
b) an effective amount of a further active ingredient that further active ingredient not being the compound according to claim 1.

16: A process for manufacturing the compound according to claim 1, or derivatives N-oxides, prodrugs, solvates, tautomers or stereoisomers thereof as well as the physiologically acceptable salts of each of the foregoing, the process comprising:
reacting
(a) a compound of formula (II) 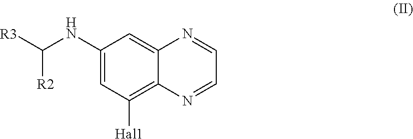
wherein
Hal1 denotes Cl, Br or I;
R2 and R3 have the same meaning as defined in claim 1 for compounds of formula (I);
under C—C coupling reaction conditions which conditions may utilize one or more suitable C—C coupling reaction reagents including catalysts
with a compound R1-RG1,
wherein
R1 has the same meaning as defined in claim 1 for compounds of formula (I);
RG1 denotes a chemical moiety being reactive under the particular C—C coupling reaction conditions utilized;
or reacting
(b) a compound of formula (III) 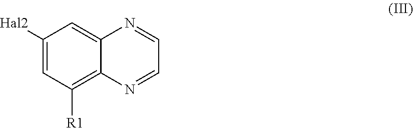
wherein
Hal2 denotes Cl, Br or I;
R1 has the same meaning as defined in claim 1 for compounds of formula (I);
under C—N coupling reaction conditions which conditions may utilize one or more suitable C—N coupling reaction reagents including catalysts
with a compound R2R3HC-NH-RG2
wherein
R2 and R3 have the same meaning as defined in claim 1 for compounds of formula (I);
RG2 denotes a chemical moiety being reactive under the particular C—N coupling reaction conditions utilized.

17: A compound of formula (II) or (III) 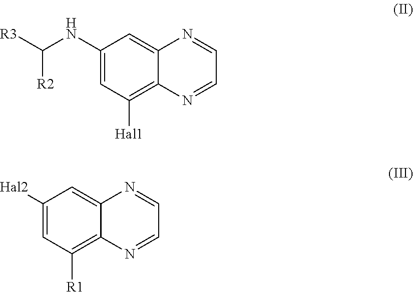
or salts thereof,
wherein
R1, R2 and R3 have the same meaning as defined in claim 1 for compounds of formula (I);
Hal1 and Hal2 both independently from each other denote Cl, Br or I.

18: A method for preventing and/or treating a medical condition, the method comprising:
administering the compound according to claim 1, or its derivatives, N-oxides, prodrugs, solvates, tautomers or stereoisomers thereof as well as the physiologically acceptable salts of each of the foregoing, including mixtures thereof in all ratios
to a subject in need thereof;
wherein said medical condition is affected by inhibiting 6-phosphofructo-2-kinase/fructose-2,6-bisphosphatase (PFKFB).

19: The method according to claim 18, wherein PFKFB is PFKFB3.

20: A method for preventing and/or treating cancer, the method comprising:
administering the compound according to claim 1, or its derivatives, N-oxides, prodrugs, solvates, tautomers or stereoisomers thereof as well as the physiologically acceptable salts of each of the foregoing, including mixtures thereof in all ratios
to a subject in need thereof.

21: The method according to claim 20, wherein the cancer is at least one member selected from the group consisting of adipose cancer, anogenital cancer, bladder cancer, breast cancer, central nervous system cancer, cervical cancer, colon cancer, connective tissue cancer, glioblastoma, glioma, kidney cancer, leukemia, lung cancer, lymphoid cancer, ovarian cancer, pancreatic cancer, prostate cancer, retinal cancer, skin cancer, stomach cancer, and uterine cancer.
